SEQUENCE LISTING (6 sequences)

SEQ ID NO: 1 - DNA, 22 bp, synthetic construct
  source 1..22: mol_type = other DNA
tgactgtgaacgttcgagatga

SEQ ID NO: 2 - DNA, 11 bp, synthetic construct
  source 1..11: mol_type = other DNA
  modified_base 3: mod_base = OTHER; note = 2'-deoxy-7-deazaguanosine
  modified_base 7: mod_base = OTHER; note = 2'-deoxy-7-deazaguanosine
  modified_base 11: mod_base = OTHER; note = 2'-deoxy-7-deazaguanosine
tcgaacgttcg

SEQ ID NO: 3 - DNA, 10 bp, synthetic construct
  source 1..10: mol_type = other DNA
aacgttcgag

SEQ ID NO: 4 - protein, 546 aa, Varicella Zoster Virus
MGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGLA

SEQ ID NO: 5 - protein, 537 aa, Varicella Zoster Virus
MGTVNKPVVGVLMGFGIITGTLRITNPVRASVLRYDDFHTDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLR

SEQ ID NO: 6 - protein, 546 aa, Varicella Zoster Virus
  VARIANT 40: note = X = I or T
  VARIANT 536: note = X = I or L
  REGION 526..546: note = Any of the amino acids in positions 526-546 can be present or absent
MGTVNKPVVGVLMGFGIITGTLRITNPVRASVLRYDDFHXDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLXRYAAWTGGLA

(19) United States
(12) Patent Application Publication
(10) Pub. No.: US 2025/0163105 A1
(43) Pub. Date: May 22, 2025

(54) IMMUNOGENICITY OF A CPG-ADJUVANTED HERPES ZOSTER VACCINE

(71) Applicant: Dynavax Technologies Corporation, Emeryville, CA (US)

(72) Inventor: Robert S. JANSSEN, Emeryville, CA (US)

(73) Assignee: Dynavax Technologies Corporation, Emeryville, CA (US)

(21) Appl. No.: 18/723,407

(22) PCT Filed: Dec. 22, 2022
(86) PCT No.: PCT/US2022/082311

§ 371 Date: Jun. 21, 2024

Related U.S. Application Data
(60) Provisional application No. 63/293,510, filed on Dec. 23, 2021.

(51) Int. Cl. C07K 14/005; A61K 39/00; A61P 31/22

(52) U.S. Cl. CPC C07K 14/005; A61P 31/22; A61K 39/00; A61K 2039/545; A61K 2039/55505; A61K 2039/55561; C12N 2710/16722; C12N 2710/16734; C12N 2710/16771

(57) ABSTRACT

The present disclosure relates to methods for increasing cell-mediated immunity against varicella zoster virus (VZV) in a human subject in need thereof by administration of an immunogenic composition comprising effective amounts of a VZV glycoprotein E antigen and an oligonucleotide comprising an unmethylated cytidine-phospho-guanosine (CpG) motif. The immunogenic compositions are suitable for prevention of herpes zoster and/or postherpetic neuralgia.

### CROSS-REFERENCE TO RELATED APPLICATION(S)

This application claims priority to and the benefit of U.S. Provisional Application No. 63/293,510, filed Dec. 23, 2021, which is incorporated herein by reference in its entirety.

### REFERENCE TO AN ELECTRONIC SEQUENCE LISTING

The content of the electronic sequence listing (377882008440SEQLIST.xml; Size: 10,729 bytes; and Date of Creation: Dec. 20, 2022) is incorporated herein by reference in its entirety.

### FIELD

The present disclosure relates to methods for increasing cell-mediated immunity against varicella zoster virus (VZV) in a human subject in need thereof by administration of an immunogenic composition comprising effective amounts of a VZV glycoprotein E antigen and an oligonucleotide comprising an unmethylated cytidine-phospho-guanosine (CpG) motif. The immunogenic compositions are suitable for prevention of herpes zoster and/or postherpetic neuralgia.

### BACKGROUND

Herpes zoster (HZ) also known as shingles is caused by reactivation of latent varicella-zoster virus (VZV) and typically manifests as a localized, dermatomal rash (Eshleman et al., Future Virology, 6(3):341-355, 2011; and Cohen, N Engl J Med, 369(3):255-263, 2013). Immunosenescence (age-dependent decrease in immunological competence) and immunodeficiency (caused by disease or medication) are the most important risk factors for developing HZ. A person's risk for herpes zoster, and complications, such as post-herpetic neuralgia and hospitalization, increases sharply after 50 years of age, closely corresponding to the age-related decline in VZV-specific T-cell-mediated immunity (CMI), which is considered important in preventing the reactivation of latent VZV and preventing the propagation of the reactivated virus (Levin et al., J Infect Dis, 197(6):825-835, 2008; Weinberg et al., J Infect Dis, 201(7):1024-1030, 2010; and Weinberg and Levin, Curr Top Microbiol Immunol, 342:341-357, 2010). HZ vaccines are believed to boost VZV-specific memory T cells, preventing their decline below the presently unknown threshold required for protection against HZ (Oxman et al., Vaccine, 29(20):3625-3627, 2011).

Prior to the availability of HZ vaccines, HZ affected nearly 1 million people in the United States annually. The incidence of HZ in unvaccinated adults in 2020 was 9.92 (95% CI, 9.82-10.01) per 1000 person-years. It generally increased with age, from 7.20/1000 person-years in the 50-54 years group to 13.99/1000 person-years in the ≥80 years group. Half of all HZ cases occur in people over 60 years of age, and it is estimated that individuals who live to be 85 years old have a 50% chance of having HZ during their lifetime (Dooling et al., MMWR, 67:103-108, 2018).

In the United States, two vaccines were available for prevention of herpes zoster in individuals who are 50 years of age or older. ZOSTAVAX® is a live attenuated virus vaccine marketed by Merck & Co., Inc. (Whitehouse Station, NJ). SHINGRIX® is a recombinant, AS01B-adjuvanted subunit vaccine marketed by GlaxoSmithKline (Research Triangle Park, NC). Although no head-to-head clinical trials were conducted, the Advisory Committee on Immunization Practices narrowly voted in 2017 to recommend preferential use of SHINGRIX® over ZOSTAVAX® based on estimates of improved efficacy against herpes zoster and postherpetic neuralgia (Dooling et al., MMWR, 67:103-108, 2018). The narrow vote in favor of SHINGRIX® (8 to 7) was associated with concerns about reactogenicity. Phase III studies for SHINGRIX® reported that 16.5% of vaccine recipients developed grade 3 adverse events versus 3.1% of placebo recipients, and that 10.8% of vaccine recipients developed grade 3 systemic events (myalgia, fatigue, headache, shivering, fever, and gastrointestinal symptoms) versus 2.4% of placebo recipients (Lal et al., N Eng J Med, 372:2087-2096, 2015; and Cunningham et al., N Eng J Med, 375:1019-1032, 2016). The relatively high levels of severe side effects after SHINGRIX® vaccination was recognized as having the potential to negatively impact second dose compliance levels in patients (Bharucha et al., Human Vaccines & Immunotherapeutics, 13:1789-1797, 2017), and this has been a persistent concern given the need for a patient to receive both doses to be protected against HZ. ZOSTAVAX® was taken off the U.S. market in 2020 as a consequence of lawsuits alleging that it caused shingles and serious zoster-related disease in some patients.

Thus, there is an unmet medical need for an HZ vaccine with a superior safety profile.

### SUMMARY

The present disclosure relates to methods for increasing cell-mediated immunity against varicella zoster virus (VZV) in a human subject in need thereof by administration of an immunogenic composition comprising effective amounts of a VZV glycoprotein E antigen and an oligonucleotide comprising an unmethylated cytidine-phospho-guanosine (CpG) motif. The immunogenic compositions are suitable for prevention of herpes zoster and/or postherpetic neuralgia.

### BRIEF DESCRIPTION OF THE DRAWINGS

FIG. 1 shows the frequency of VZV gE-reactive CD4+ T cells expressing 2, 3 and 4 markers of activation after incubation of PBMCs in the presence of a series of overlapping peptides spanning VZV gE, with non-stimulated (background) response subtracted. PBMCs were isolated from blood samples obtained from study subjects at Week 0 (baseline).

FIG. 2 shows the frequency of VZV gE-reactive CD4+ T cells expressing 2, 3 and 4 markers of activation after incubation of PBMCs in the presence of a series of overlapping peptides spanning VZV gE, with non-stimulated (background) response subtracted. PBMCs were isolated from blood samples obtained from study subjects at Week 8 (˜4 weeks after first injection).

FIG. 3 shows the frequency of VZV gE-reactive CD4+ T cells expressing 2, 3 and 4 markers of activation after incubation of PBMCs in the presence of a series of overlapping peptides spanning VZV gE, with non-stimulated (background) response subtracted. PBMCs were isolated from blood samples obtained from study subjects at Week 12 (˜4 weeks after second injection).

### DETAILED DESCRIPTION

The present disclosure relates to methods for increasing cell-mediated immunity against varicella zoster virus (VZV) in a human subject in need thereof by administration of an immunogenic composition comprising effective amounts of a VZV glycoprotein E antigen and an oligonucleotide comprising an unmethylated cytidine-phospho-guanosine (CpG) motif. The immunogenic compositions are suitable for prevention of herpes zoster (HZ) and/or postherpetic neuralgia (PHN).

Reactogenicity of protein subunit vaccines can be influenced by a variety of factors, chief among which is the nature of the adjuvant included to improve immunogenicity. Different Adjuvant Systems (AS) adjuvants combined with a model protein antigen were compared by immunization of healthy, human subjects. The AS01B adjuvant was found to induce higher levels of local and systemic reactogenicity than the comparator adjuvants (AS01E, AS03A, AS04, and alum)(Leroux-Roels et al., Clin Immunol, 169:16-27, 2016). This suggests that AS01B is contributing significantly to the concerning reactogenicity profile of SHINGRIX® (a recombinant, AS01B-adjuvanted subunit zoster vaccine marketed by GlaxoSmithKline, Research Triangle Park, NC).

Z-1018 is an exemplary non-infectious vaccine, which is being developed for active booster immunization for prevention of herpes zoster (HZ) (shingles) as 2 intramuscular (IM) doses administered 2 months apart in individuals 50-69 years of age as described in Example 1. Z-1018 is comprised of a glycoprotein E (gE) antigen, and adjuvanted by mixing with CpG 1018@adjuvant with and without aluminum hydroxide (alum). CpG 1018@adjuvant (Dynavax Technologies Corporation) is a synthetic 22-base phosphorothioate oligodeoxynucleotide (PS ODN) containing a cytidine-phospho-guanosine (CpG) immunostimulatory sequence that is an agonist for Toll-like receptor 9 (TLR9).

### General Techniques and Definitions

The practice of the present disclosure will employ, unless otherwise indicated, conventional techniques of molecular biology (including recombinant techniques), microbiology, cell biology, biochemistry and immunology, which are within the skill of the art.

As used herein and in the appended claims, the singular forms “a”, “an”, and “the” include plural references unless indicated otherwise. For example, “an” excipient includes one or more excipients.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

The term “about” as used herein in reference to a value, encompasses from 90% to 110% of that value (e.g., about 3000 μg of CpG 1018@adjuvant refers to 2700 μg to 3300 μg of CpG 1018® adjuvant).

As used interchangeably herein, the terms “polynucleotide” and “oligonucleotide” include single-stranded DNA (ssDNA), double-stranded DNA (dsDNA), single-stranded RNA (ssRNA) and double-stranded RNA (dsRNA), modified oligonucleotides and oligonucleosides or combinations thereof. The oligonucleotide can be linearly or circularly configured, or the oligonucleotide can contain both linear and circular segments. Oligonucleotides are polymers of nucleosides joined, generally, through phosphodiester linkages, although alternate linkages, such as phosphorothioate esters may also be used in oligonucleotides. A nucleoside consists of a purine (adenine (A) or guanine (G) or derivative thereof) or pyrimidine (thymine (T), cytosine (C) or uracil (U), or derivative thereof) base bonded to a sugar. The four nucleoside units (or bases) in DNA are called deoxyadenosine, deoxyguanosine, thymidine, and deoxycytidine. A nucleotide is a phosphate ester of a nucleoside.

The terms “CpG,” “CpG motif,” and “cytosine-phosphate-guanosine,” as used herein, refer to an unmethylated cytidine-phospho-guanosine dinucleotide, which when present in an oligonucleotide contributes to a measurable immune response in vitro, in vivo and/or ex vivo. Examples of measurable immune responses include, but are not limited to, antigen-specific antibody production, secretion of cytokines, activation or expansion of lymphocyte populations, such as NK cells, CD4+T lymphocytes, CD8+T lymphocytes, B lymphocytes, and the like. Preferably, the CpG oligonucleotide preferentially activates a Th1-type response.

An “effective amount” or a “sufficient amount” of a substance is that amount sufficient to effect beneficial or desired results, including clinical results, and, as such, an “effective amount” depends upon the context in which it is being applied. In the context of administering an immunogenic composition, an effective amount contains sufficient antigen and TLR9 agonist to stimulate an immune response (preferably a seroprotective level of antibody to the antigen).

In the present disclosure, the terms “individual” and “subject” refer to a human.

The term “dose” as used herein in reference to an immunogenic composition refers to a measured portion of the immunogenic composition taken by (administered to or received by) a subject at any one time.

The terms “isolated” and “purified” as used herein refers to a material that is removed from at least one component with which it is naturally associated (e.g., removed from its original environment). The term “isolated,” when used in reference to a recombinant protein, refers to a protein that has been removed from the culture medium of the host cell that produced the protein.

“Stimulation” of a response or parameter includes eliciting and/or enhancing that response or parameter when compared to otherwise same conditions except for a parameter of interest, or alternatively, as compared to another condition (e.g., increase in TLR-signaling in the presence of a TLR agonist as compared to the absence of the TLR agonist). For example, “stimulation” of an immune response means an increase in the response. Depending upon the parameter measured, the increase may be from 5-fold to 500-fold or over, or from 5, 10, 50, or 100-fold to 500, 1,000, 5,000, or 10,000-fold.

As used herein the term “immunization” refers to a process that increases a mammalian subject's reaction to antigen and therefore improves its ability to resist or overcome infection and/or resist disease.

The term “vaccination” as used herein refers to the introduction of vaccine into a body of a mammalian subject.

“Adjuvant” refers to a substance which, when added to a composition comprising an antigen, enhances or potentiates an immune response to the antigen in the mammalian recipient upon exposure.

### I. Immunogenic Compositions and Kits

The present disclosure relates to immunogenic compositions for stimulating an immune response against varicella zoster virus (VZV), comprising a VZV glycoprotein E (gE) antigen and a toll-like receptor 9 (TLR9) agonist, wherein the TLR9 agonist is an oligonucleotide of from 8 to 35 nucleotides in length comprising an unmethylated cytidine-phospho-guanosine (also referred to as CpG or cytosine-phosphate-guanosine) motif, and the gE antigen and oligonucleotide are present in the immunogenic composition in amounts effective to stimulate an immune response against the gE antigen in a human subject. In some embodiments, the immunogenic compositions further comprise an aluminum salt adjuvant to which the VZV gE antigen is adsorbed. In some preferred embodiments, the present disclosure relates to immunogenic compositions for increasing cell-mediated immunity against VZV.

In particular, the present disclosure relates to immunogenic compositions comprising: i) an unmethylated cytidine-phospho-guanosine (CpG)-containing oligonucleotide comprising the sequence of 5′-TGACTGTGAA CGTTCGAGAT GA-3′(SEQ ID NO: 1); ii) a truncated VZV glycoprotein E (gE) antigen; and iii) at least one excipient, wherein the gE antigen is a recombinant protein produced in mammalian cells and is present in the immunogenic composition in an amount of from about 25 μg to about 150 μg, the oligonucleotide is present in the immunogenic composition in an amount of from about 750 μg to about 6000 μg, and the at least one excipient comprises pharmaceutically acceptable buffer.

In exemplary embodiments, the immunogenic compositions comprise i) an unmethylated cytidine-phospho-guanosine (CpG)-containing oligonucleotide comprising the sequence of 5′-TGACTGTGAA CGTTCGAGAT GA-3′(SEQ ID NO: 1); ii) a truncated VZV glycoprotein E (gE) antigen; and iii) at least one excipient, wherein the gE antigen is a recombinant protein produced in mammalian cells and is present in the immunogenic composition in an amount of about 100 μg, the oligonucleotide is present in the immunogenic composition in an amount of about 3000 μg or about 6000 μg, and the at least one excipient comprises pharmaceutically acceptable buffer.

### A. Oligonucleotide Toll-Like Receptor 9 (TLR9) Agonists

Toll-like receptors (TLRs) are expressed in and on dendritic cells and other innate immune cells and are among the most important receptors for stimulating a response to the presence of invading pathogens. Humans have multiple types of TLRs that are similar in structure but recognize different parts of viruses or bacteria. By activating specific TLRs, it is possible to stimulate and control specific types of innate immune responses that can be harnessed to enhance adaptive responses.

TLR9 (CD289) recognizes unmethylated cytidine-phospho-guanosine (CpG) motifs found in microbial DNA, which can be mimicked using synthetic CpG-containing oligodeoxynucleotides (CpG-ODNs). CpG-ODNs are known to enhance antibody production and to stimulate T helper 1 (Th1) cell responses (Coffman et al., Immunity, 33:492-503, 2010).

Based on structure and biological function, CpG-ODNs have been divided into three general classes: CpG-A, CpG-B, and CpG-C(Campbell, Methods Mol Biol, 1494:15-27, 2017). The degree of B cell activation varies between the classes with CpG-A ODNs being weak, CpG-C ODNs being good, and CpG-B ODNs being strong B cell activators. Oligonucleotide TLR9 agonists of the present disclosure are preferably good B cell activators (CpG-C ODN) or more preferably strong (CpG-B ODN) B cell activators.

Optimal oligonucleotide TLR9 agonists often contain a palindromic sequence following the general formula of: 5′-purine-purine-CG-pyrimidine-pyrimidine-3′, or 5′-purine-purine-CG-pyrimidine-pyrimidine-CG-3′ (U.S. Pat. No. 6,589,940). TLR9 agonism is also observed with certain non-palindromic CpG-enriched phosphorothioate oligonucleotides, but may be affected by changes in the nucleotide sequence. Additionally, TLR9 agonism is abolished by methylation of the cytosine within the CpG dinucleotide. Accordingly in some embodiments, the TLR9 agonist is an oligonucleotide of from 8 to 35 nucleotides in length comprising the sequence 5′-AACGTTCG-3′. In some embodiments, the oligonucleotide is greater than 8, 9, 10, 11, 12, 13, 14, 15, 16, 17, 18, 19, or 20 nucleotides in length, and the oligonucleotide is less than 35, 34, 33, 32, 31, 30, 29, 28, 27, 26, 25, or 24 nucleotides in length. In some embodiments, the TLR9 agonist is an oligonucleotide of from 10 to 35 nucleotides in length comprising the sequence 5′-AACGTTCGAG-3′ (SEQ ID NO:3). In some embodiments, the oligonucleotide is greater than 10, 11, 12, 13, 14, 15, 16, 17, 18, 19, or 20 nucleotides in length, and the oligonucleotide is less than 35, 34, 33, 32, 31, 30, 29, 28, 27, 26, 25, or 24 nucleotides in length.

Researchers at Dynavax Technologies Corporation (Emeryville, CA) have identified a 22-mer phosphorothioate linked oligodeoxynucleotide, CpG 1018@adjuvant, which contains specific sequences that can substantially enhance the immune response to co-administered antigens across species (Campbell, Methods Mol Biol, 1494:15-27, 2017). CpG 1018® adjuvant (5′-TGACTGTGAA CGTTCGAGAT GA-3′, set forth as SEQ ID NO:1) was chosen after screening a broad panel of oligonucleotides for immunostimulatory activity in vitro and in vivo. CpG 1018® adjuvant is a CpG-B ODN that is active in mice, rabbits, dogs, baboons, cynomolgus monkeys, and humans. Thus in some preferred embodiments, the TLR9 agonist is an oligonucleotide of from 22 to 35 nucleotides in length comprising the sequence of SEQ ID NO:1.

Although the exemplary oligonucleotide TLR9 agonist, CpG 1018® adjuvant, is a CpG-ODN, the present disclosure is not restricted to fully DNA molecules. That is, in some embodiments, the TLR9 agonist is a DNA/RNA chimeric molecule in which the CpG(s) and the palindromic sequence are deoxyribonucleic acids and one or more nucleic acids outside of these regions are ribonucleic acids. In some embodiments, the CpG oligonucleotide is linear. In other embodiments, the CpG oligonucleotide is circular or includes hairpin loop(s). The CpG oligonucleotide may be single stranded or double stranded.

In some embodiments, the CpG oligonucleotide may contain modifications. Modifications include but are not limited to, modifications of the 3′OH or 5′OH group, modifications of the nucleotide base, modifications of the sugar component, and modifications of the phosphate group. Modified bases may be included in the palindromic sequence of the CpG oligonucleotide as long as the modified base(s) maintains the same specificity for its natural complement through Watson-Crick base pairing (e.g., the palindromic portion is still self-complementary). In some embodiments, the CpG oligonucleotide comprises a non-canonical base. In some embodiments, the CpG oligonucleotide comprises a modified nucleoside. In some embodiments, the modified nucleoside is selected from the group consisting of 2′-deoxy-7-deazaguanosine, 2′-deoxy-6-thioguanosine, arabinoguanosine, 2′-deoxy-2′substituted-arabinoguanosine, and 2′-O-substituted-arabinoguanosine. In some embodiments, the TLR9 agonist is an oligonucleotide comprising the sequence 5′-TCG₁AACG₁TTCG₁-3′ (SEQ ID NO:2), in which G₁ is 2′-deoxy-7-deazaguanosine. In some embodiments, the oligonucleotide comprises the sequence 5′-TCG₁AACG₁TTCG₁-X-G₁CTTG₁CAAG₁CT-5′, and in which G₁ is 2′-deoxy-7-deazaguanosine and X is glycerol (5′-SEQ ID NO:2-3′-X-3′-SEQ ID NO:2-5′).

The CpG oligonucleotide may contain a modification of the phosphate group. For example, in addition to phosphodiester linkages, phosphate modifications include, but are not limited to, methyl phosphonate, phosphorothioate, phosphoramidate (bridging or non-bridging), phosphotriester and phosphorodithioate and may be used in any combination. Other non-phosphate linkages may also be used. In some embodiments, the oligonucleotides comprise only phosphorothioate backbones. In some embodiments, the oligonucleotides comprise only phosphodiester backbones. In some embodiments, the oligonucleotide comprises a combination of phosphate linkages in the phosphate backbone such as a combination of phosphodiester and phosphorothioate linkages. Oligonucleotides with phosphorothioate backbones can be more immunogenic than those with phosphodiester backbones and appear to be more resistant to degradation after injection into the host (Braun et al., J Immunol, 141:2084-2089, 1988; and Latimer et al., Mol Immunol, 32:1057-1064, 1995). The CpG oligonucleotides of the present disclosure include at least one, two or three internucleotide phosphorothioate ester linkages. In some embodiments, when a plurality of CpG oligonucleotide molecules are present in a pharmaceutical composition comprising at least one excipient, both stereoisomers of the phosphorothioate ester linkage are present in the plurality of CpG oligonucleotide molecules. In some embodiments, all of the internucleotide linkages of the CpG oligonucleotide are phosphorothioate linkages, or said another way, the CpG oligonucleotide has a phosphorothioate backbone.

A unit dose of the immunogenic composition, which in exemplary embodiments is a 1.0 ml dose, may comprises from about 750 μg to about 6000 μg of the CpG oligonucleotide, preferably about 3000 μg or about 6000 μg of the CpG oligonucleotide. In some embodiments, a 1.0 ml dose of the immunogenic composition comprises greater than or equal to about 750, 1000, 1250 or 1500 μg of the CpG oligonucleotide, and less than or equal to about 6000, 5000, 4000, or 3000 μg of the CpG oligonucleotide. In some embodiments, a 1.0 ml dose of the immunogenic composition comprises about 750, 1500, 3000 or 6000 μg of the CpG oligonucleotide. In some embodiments, a 1.0 ml dose of the immunogenic composition comprises about 3000 μg of the CpG oligonucleotide. In some embodiments, a 1.0 ml dose of the immunogenic composition comprises about 6000 μg of the CpG oligonucleotide.

The CpG oligonucleotides described herein are in their pharmaceutically acceptable salt form unless otherwise indicated. Exemplary basic salts include ammonium salts, alkali metal salts such as sodium, lithium, and potassium salts, alkaline earth metal salts such as calcium and magnesium salts, zinc salts, salts with organic bases (for example, organic amines) such as N-Me-D-glucamine, N-[1-(2,3-dioleoyloxy)propyl]-N,N,N-trimethylammonium chloride, choline, tromethamine, dicyclohexylamines, t-butyl amines, and salts with amino acids such as arginine, lysine and the like. In some embodiment, the CpG oligonucleotides are in the ammonium, sodium, lithium, or potassium salt form. In one preferred embodiment, the CpG oligonucleotides are in the sodium salt form.

### B. Varicella Zoster Virus (VZV) Glycoprotein E (gE) Antigen

A VZV gE antigen of the immunogenic compositions of the present disclosure comprises gE or a fragment thereof. In preferred embodiments, the gE antigen is recognized by VZV-reactive antibodies and/or peptide fragments of gE are recognized by VZV-reactive T cells. In some embodiments, the gE antigen is a recombinant protein, while in other embodiments the gE antigen is a purified from VZV virions. In some preferred embodiments, the gE antigen is an isolated antigen. In some embodiments, the gE antigen is not a fusion protein. In some preferred embodiments, the gE antigen of the immunogenic compositions of the present disclosure is not part of a live attenuated VZV or a whole inactivated VZV.

In some embodiments, the VZV gE antigen is a truncated recombinant protein devoid of transmembrane and intravirion domains of a full-length VZV gE antigen. In some embodiments, the recombinant protein is produced in mammalian cells, such as Chinese hamster ovary (CHO) cells. The amino acid sequence of a representative gE is set forth as GenBank No. AQT34120.1. In some embodiments, the gE antigen comprises the amino acid sequence from residues 39-585 of GenBank No. AQT34120.1, or the amino acid sequence that is at least 90%, 95%, 96%, 97%, 98% or 99% identical thereto.

In some embodiments, the amino acid sequence of the gE is set forth as SEQ ID NO:4:

MGTVNKPVVG VLMGFGIITG TLRITNPVRA SVLRYDDFHI
DEDKLDTNSV YEPYYHSDHA ESSWVNRGES SRKAYDHNSP
YIWPRNDYDG FLENAHEHHG VYNQGRGIDS GERLMQPTQM
SAQEDLGDDT GIHVIPTLNG DDRHKIVNVD QRQYGDVFKG
DLNPKPQGQR LIEVSVEENH PFTLRAPIQR IYGVRYTETW
SFLPSLTCTG DAAPAIQHIC LKHTTCFQDV VVDVDCAENT
KEDQLAEISY RFQGKKEADQ PWIVVNTSTL FDELELDPPE
IEPGVLKVLR TEKQYLGVYI WNMRGSDGTS TYATFLVTWK
GDEKTRNPTP AVTPQPRGAE FHMWNYHSHV FSVGDTFSLA
MHLQYKIHEA PFDLLLEWLY VPIDPTCQPM RLYSTCLYHP
NAPQCLSHMN SGCTFTSPHL AQRVASTVYQ NCEHADNYTA
YCLGISHMEP SFGLILHDGG TTLKFVDTPE SLSGLYVFVV
YFNGHVEAVA YTVVSTVDHF VNAIEERGFP PTAGQPPATT
KPKEITPVNP GTSPLIRYAA WTGGLA.

In some embodiments, the gE antigen comprises the amino acid sequence of SEQ ID NO:4, or the amino acid sequence that is at least 90%, 95%, 96%, 97%, 98% or 99% identical to SEQ ID NO:4.

In some embodiments, the amino acid sequence of the gE is set forth as SEQ ID NO:5:

MGTVNKPVVG VLMGFGIITG TLRITNPVRA SVLRYDDFHT
DEDKLDTNSV YEPYYHSDHA ESSWVNRGES SRKAYDHNSP
YIWPRNDYDG FLENAHEHHG VYNQGRGIDS GERLMQPTQM
SAQEDLGDDT GIHVIPTLNG DDRHKIVNVD QRQYGDVFKG
DLNPKPQGQR LIEVSVEENH PFTLRAPIQR IYGVRYTETW
SFLPSLTCTG DAAPAIQHIC LKHTTCFQDV VVDVDCAENT
KEDQLAEISY RFQGKKEADQ PWIVVNTSTL FDELELDPPE
IEPGVLKVLR TEKQYLGVYI WNMRGSDGTS TYATFLVTWK
GDEKTRNPTP AVTPQPRGAE FHMWNYHSHV FSVGDTFSLA
MHLQYKIHEA PFDLLLEWLY VPIDPTCQPM RLYSTCLYHP
NAPQCLSHMN SGCTFTSPHL AQRVASTVYQ NCEHADNYTA
YCLGISHMEP SFGLILHDGG TTLKFVDTPE SLSGLYVFVV
YFNGHVEAVA YTVVSTVDHF VNAIEERGFP PTAGQPPATT
KPKEITPVNP GTSPLLR.

In some embodiments, the gE antigen comprises the amino acid sequence of SEQ ID NO:5, or the amino acid sequence that is at least 90%, 95%, 96%, 97%, 98% or 99% identical to SEQ ID NO:5.

In some embodiments, the amino acid sequence of the gE corresponds to a truncated gE (see, U.S. Publication No. 2021/0187099). In some embodiments, the amino acid sequence of the gE is set forth as SEQ ID NO:6:

MGTVNKPVVG VLMGFGIITG TLRITNPVRA SVLRYDDFHX
DEDKLDTNSV YEPYYHSDHA ESSWVNRGES SRKAYDHNSP
YIWPRNDYDG FLENAHEHHG VYNQGRGIDS GERLMQPTQM
SAQEDLGDDT GIHVIPTLNG DDRHKIVNVD QRQYGDVFKG
DLNPKPQGQR LIEVSVEENH PFTLRAPIQR IYGVRYTETW
SFLPSLTCTG DAAPAIQHIC LKHTTCFQDV VVDVDCAENT
KEDQLAEISY RFQGKKEADQ PWIVVNTSTL FDELELDPPE
IEPGVLKVLR TEKQYLGVYI WNMRGSDGTS TYATFLVTWK
GDEKTRNPTP AVTPQPRGAE FHMWNYHSHV FSVGDTFSLA
MHLQYKIHEA PFDLLLEWLY VPIDPTCQPM RLYSTCLYHP
NAPQCLSHMN SGCTFTSPHL AQRVASTVYQ NCEHADNYTA
YCLGISHMEP SFGLILHDGG TTLKFVDTPE SLSGLYVFVV
YFNGHVEAVA YTVVSTVDHF VNAIEERGFP PTAGQPPATT
KPKEIXXXXX XXXXXXXXXX XXXXXX,

wherein
X at position 40 is I or T,
X at any one of positions 526-546 is present or missing,
X at position 526 if present is T,
X at position 527 if present is P,
X at position 528 if present is V,
X at position 529 if present is N,
X at position 530 if present is P,
X at position 531 if present is G,
X at position 532 if present is T,
X at position 533 if present is S,
X at position 534 if present is P,
X at position 535 if present is L,
X at position 536 if present is I or L,
X at position 537 if present is R,
X at position 538 if present is Y,
X at position 539 if present is A,
X at position 540 if present is A,
X at position 541 if present is W,
X at position 542 if present is T,
X at position 543 if present is G,
X at position 544 if present is G,
X at position 545 if present is L,
X at position 546 if present is A,
such that the amino acid sequence of SEQ ID NO:6 is 525, 526, 527, 528, 529, 530, 531, 532, 533, 534, 535, 536, 537, 538, 539, 540, 541, 542, 543, 544, 545 or 546 amino acids in length. In some embodiments, the gE antigen comprises the amino acid sequence of SEQ ID NO:6, or the amino acid sequence that is at least 90%, 95%, 96%, 97%, 98% or 99% identical to SEQ ID NO:6.

A unit dose of the immunogenic composition, which in exemplary embodiments is a 1.0 ml dose, may comprise from about 15 μg to about 150 μg of the gE antigen, preferably from about 25 μg to about 125 μg of the gE antigen, preferably about 80 to about 120 μg of the gE antigen, or about 100 μg of the gE antigen. In some embodiments, a 1.0 ml unit dose of the immunogenic composition, may comprise about 25 μg, about 50 μg, about 75 μg, about 100 μg, about 125 μg, or about 150 μg of the gE antigen.

### C. Additional Components

The immunogenic compositions of the present disclosure may comprise one or more additional components, such as one or more excipients, another adjuvant, and/or additional antigens.

### 1. Excipients

Pharmaceutically acceptable excipients of the present disclosure include for instance, solvents, bulking agents, buffering agents, tonicity adjusting agents, and preservatives (Pramanick et al., Pharma Times, 45:65-77, 2013). In some embodiments the immunogenic compositions may comprise an excipient that functions as one or more of a solvent, a bulking agent, a buffering agent, and a tonicity adjusting agent (e.g., sodium chloride in saline may serve as both an aqueous vehicle and a tonicity adjusting agent).

In some embodiments, the immunogenic compositions comprise an aqueous vehicle as a solvent. Suitable vehicles include for instance sterile water, saline solution, phosphate buffered saline, and Ringer's solution. In some embodiments, the composition is isotonic.

The immunogenic compositions may comprise a buffering agent. Buffering agents control pH to inhibit degradation of the active agent during processing, storage and optionally reconstitution. Suitable buffers include for instance salts comprising acetate, citrate, phosphate, sulfate or Tris. In preferred embodiments, the buffer is not a phosphate-containing buffer. In some preferred embodiments, the buffer is a Tris buffer. Other suitable buffers include for instance amino acids such as arginine, glycine, histidine, and lysine. The buffering agent may further comprise hydrochloric acid or sodium hydroxide. In some embodiments, the buffering agent maintains the pH of the composition within a range of 6 to 9. In some embodiments, the pH is greater than (lower limit) 6, 7 or 8. In some embodiments, the pH is less than (upper limit) 9, 8, or 7. That is, the pH is in the range of from about 6 to 9 in which the lower limit is less than the upper limit. In some embodiments, the pH is about 6.5, about 7.0, or about 7.5.

The immunogenic compositions may comprise a tonicity adjusting agent. Suitable tonicity adjusting agents include for instance dextrose, glycerol, sodium chloride, glycerin and mannitol.

The immunogenic compositions may comprise a bulking agent. Bulking agents are particularly useful when the pharmaceutical composition is to be lyophilized before administration. In some embodiments, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The immunogenic compositions may comprise a preservative. Suitable preservatives include for instance antioxidants and antimicrobial agents. However, in preferred embodiments, the immunogenic composition is prepared under sterile conditions and is in a single use container, and thus does not necessitate inclusion of a preservative.

### 2. Additional Adjuvants

Adjuvants are known in the art and include, but are not limited to, alum (aluminum salts), oil-in-water emulsions, water-in-oil emulsions, liposomes, and microparticles, such as poly(lactide-co-glycolide) microparticles (Shah et al., Methods Mol Biol, 1494:1-14, 2017). In some embodiments, the immunogenic compositions further comprises an aluminum salt adjuvant to which the gE antigen is adsorbed. In some embodiments, the aluminum salt adjuvant comprises one or more of the group consisting of amorphous aluminum hydroxyphosphate sulfate, aluminum hydroxide, aluminum phosphate, and potassium aluminum sulfate. In some embodiments, the aluminum salt adjuvant comprises one or both of aluminum hydroxide and aluminum phosphate. In some embodiments, the aluminum salt adjuvant consists of aluminum hydroxide.

In some embodiments, a unit dose, which in exemplary embodiments is a 1.0 ml dose of the immunogenic composition comprises from about 0.25 to about 1.25 mg Al³⁺, preferably from about 0.50 to about 1.00 mg Al³⁺. In some embodiments, the immunogenic composition comprises about 0.50 mg, about 0.75 mg, or about 1.00 mg Al³⁺, preferably about 0.75 mg Al³⁺.

In other embodiments, the immunogenic composition further comprises an additional adjuvant. Additional suitable adjuvants include, but are not limited to, squalene-in-water emulsions (e.g., MF59 or AS03), TLR3 agonists (e.g., poly-IC or poly-ICLC), TLR5 agonists (bacterial flagellin), and TLR7 and/or TLR8 agonists (imidazoquinoline derivatives such as imiquimod, and resiquimod)(Coffman et al., Immunity, 33:492-503, 2010). For veterinary use and for production of antibodies in non-human animals, mitogenic components of Freund's adjuvant (both complete and incomplete) can be used. However, in preferred embodiments, the immunogenic composition does not comprise one or more of a saponin, a TLR4 agonist and a liposome. In some preferred embodiments, the immunogenic composition does not comprise one or more of QS21, 3-O-deacylated monophosphoryl lipid A (3D-MPL1), dioleoyl phosphatidylcholine, and/or cholesterol.

### D. Kits

The present disclosure also provides kits comprising: i) an immunogenic composition comprising a VZV gE antigen and a CpG oligonucleotide; and ii) a set of instructions for administration of the immunogenic composition to stimulate an immune response against the gE antigen in a human subject in need thereof. Additionally, the present disclosure provides kits comprising: i) a first composition comprising a VZV gE antigen; ii) a second composition comprising a CpG oligonucleotide; iii) instructions for mixing the first composition with the second composition to prepare an immunogenic composition; and optionally iv) a further set of instructions for administration of the immunogenic composition to stimulate an immune response against the gE antigen in a human subject in need thereof. In some preferred embodiments, the CpG oligonucleotide comprises the sequence of 5′-TGACTGTGAA CGTTCGAGAT GA-3′ (SEQ ID NO:1). In some preferred embodiments, stimulating an immune response against the gE antigen comprises increasing cell-mediated immunity against VZV in the human subject.

In still further embodiments, the present disclosure provides kits comprising: i) a first composition comprising a VZV gE antigen; ii) a second composition comprising a CpG oligonucleotide; iii) a third composition comprising an aluminum salt adjuvant, iv) instructions for prepare an immunogenic composition from the first, second and third compositions; and optionally v) a further set of instructions for administration of the immunogenic composition to stimulate an immune response against the gE antigen in a human subject in need thereof. In some preferred embodiments, the CpG oligonucleotide comprises the sequence of 5′-TGACTGTGAA CGTTCGAGAT GA-3′ (SEQ ID NO:1). In some preferred embodiments, stimulating an immune response against the gE antigen comprises increasing cell-mediated immunity against VZV in the human subject.

The kits may comprise an immunogenic composition packaged appropriately. For example, if the immunogenic composition is a freeze-dried power, a vial with a resilient stopper is normally used so that the powder may be easily resuspended by injecting fluid (e.g., sterile water, saline, etc.) through the resilient stopper. In some embodiments, the kits comprise a device for administration (e.g., syringe and needle). The instructions relating to the use of the immunogenic composition generally include information as to dosage, schedule and route of administration for the intended methods of use. In some embodiments, the immunogenic compositions are for stimulating an immune response against VZV, such as for increasing cell-mediated immunity against VZV in the human subject.

### II. Methods of Use

The present disclosure relates to methods for stimulating an immune response against VZV, comprising: administering an immunogenic composition comprising a VZV glycoprotein E (gE) antigen and a CpG oligonucleotide, to a human subject so as to stimulate an immune response against the gE antigen in the human subject. In preferred embodiments, the immunogenic compositions are to be administered by intramuscular injection, optionally in a volume of about 1.0 mL (e.g., unit dose). In some embodiments, the intramuscular injection is into the deltoid muscle of the upper arm of a human subject in need thereof.

Stimulating an immune response, means increasing the immune response, which can arise from eliciting a de novo immune response (e.g., as a consequence of an initial vaccination regimen) or enhancing an existing immune response (e.g., as a consequence of a booster vaccination regimen). In some embodiments, stimulating an immune response includes but is not limited to one or more of the group consisting of: stimulating cytokine production; stimulating B lymphocyte proliferation; stimulating antibody production; stimulating interferon pathway-associated gene expression; stimulating chemoattractant-associated gene expression; and stimulating plasmacytoid dendritic cell maturation.

In some preferred embodiments, the methods of the present disclosure are suitable for increasing cell-mediated immunity against VZV in the subject relative to cell-mediated immunity against VZV in the subject prior to administration of the immunogenic composition. In some embodiments, the increase in cell-mediated immunity against VZV comprises an increase in frequency of gE antigen-reactive, activated CD4+ T cells, wherein the activated CD4+ T cells express two or more activation markers selected from interleukin-2, interferon-gamma, tumor necrosis factor-alpha, and CD40L (CD154). In some embodiments, cell-mediated immunity is measured by flow cytometry. In some embodiments, the gE antigen-reactive, activated CD4+ T cells are present in peripheral blood mononuclear cells of the subject.

In some preferred embodiments, the methods of the present disclosure are suitable for increasing both of cell-mediated immunity against VZV in the subject, and humoral immunity against VZV in the subject. In some embodiments, administration of the immunogenic composition results in an increase in humoral immunity against VZV in the subject relative to humoral immunity against VZV in the subject prior to administration of the immunogenic composition. In some embodiments, the increase in humoral immunity against VZV comprises an increased concentration of gE antigen-reactive IgG. In some embodiments, the gE antigen-reactive IgG is measured by ELISA. In some embodiments, the increase in humoral immunity against VZV comprises an increased concentration of VZV-reactive antibodies. In some embodiments, the increased concentration of VZV-reactive antibodies comprises an increase in concentration of gE- and/or VZV-neutralizing antibodies.

The human subject is typically 18 years of age or older. In some embodiments, the human subject is a healthy male or female adult. In some embodiments, the human subject is 50 years of age or older. In some embodiments, the human subject is 50-69 years of age. In some embodiments, the human subject is suspected to have a latent VZV infection. In some embodiments, the human subject contracted varicella as a child (12 years of age or younger), adolescent (13-17 years of age), or a young adult (18-25 years of age), prior to administration of the immunogenic composition. In other embodiments, the human subject is not infected with VZV or was not known to have been infected with VZV. In some embodiments, the human subject was not previously vaccinated against varicella or herpes zoster. In some embodiments, the human subject is 18 years of age or older and is suffering from an immunodeficiency or immunosuppression caused by known disease or therapy.

In some methods of the present disclosure, a first dose and a second dose of the immunogenic composition is administered to the human subject, with the second dose administered from 1 month to 1 year after the first dose. In some embodiments, the second dose is administered from 2 months to 6 months after the first dose. In some embodiments, the second dose is administered about 2 months after the first dose. In exemplary embodiments each dose is a 1-mL dose administered by intramuscular injection.

### Enumerated Embodiments

1. A method of increasing cell-mediated immunity against varicella zoster virus (VZV) in a human subject in need thereof, the method comprising:
administering to the human subject an immunogenic composition comprising: i) an unmethylated cytidine-phospho-guanosine (CpG)-containing oligonucleotide comprising the sequence of 5′-TGACTGTGAA CGTTCGAGAT GA-3′(SEQ ID NO: 1); and ii) a truncated VZV glycoprotein E (gE) antigen, which are present in the immunogenic composition in amounts effective to increase cell-mediated immunity against VZV relative to cell-mediated immunity against VZV in the subject prior to administration of the immunogenic composition.

2. The method of embodiment 1, wherein the immunogenic composition comprises: from about 25 μg to about 150 μg of the gE antigen, and from about 750 μg to about 6000 μg of the oligonucleotide, optionally wherein the immunogenic composition comprises from about 50 μg to about 100 μg of the gE antigen, and from about 1500 μg to about 6000 μg of the oligonucleotide.

3. The method of embodiment 1, wherein the immunogenic composition comprises about 25 μg, about 50 μg, about 75 μg, about 100 μg, about 125 μg, or about 150 μg of the gE antigen, and about 750 μg, about 1500 μg, about 3000 μg, or about 6000 μg of the oligonucleotide.

4. The method of embodiment 3, wherein the immunogenic composition comprises about 100 μg of the gE antigen, and about 3000 μg or 6000 μg of the oligonucleotide.

5. A method of increasing an immune response against varicella zoster virus (VZV) in a human subject in need thereof, the method comprising:
administering to the human subject an immunogenic composition comprising: i) an unmethylated cytidine-phospho-guanosine (CpG)-containing oligonucleotide comprising the sequence of 5′-TGACTGTGAA CGTTCGAGAT GA-3′(SEQ ID NO: 1); and ii) a truncated VZV glycoprotein E (gE) antigen, wherein the immunogenic composition comprises about 100 mcg of the gE antigen and about 3000 mcg or about 6000 mcg of the oligonucleotide.

6. The method of any one of embodiments 1-5, wherein the oligonucleotide comprises at least one phosphorothioate linkage, or wherein all nucleotide linkages are phosphorothioate linkages.

7. The method of any one of embodiments 1-6, wherein the oligonucleotide is a single-stranded oligodeoxynucleotide.

8. The method of any one of embodiments 1-7, wherein the gE antigen is a recombinant protein devoid of transmembrane and intravirion domains of a full-length VZV gE antigen.

9. The method of embodiment 8, wherein the recombinant protein is produced in mammalian cells.

10. The method of any one of embodiments 1-9, wherein the immunogenic composition further comprises an aluminum salt adjuvant.

11. The method of embodiment 10, wherein the aluminum salt adjuvant comprises one or more of the group consisting of amorphous aluminum hydroxyphosphate sulfate, aluminum hydroxide, aluminum phosphate, and potassium aluminum sulfate.

12. The method of embodiment 10, wherein the aluminum salt adjuvant comprises aluminum hydroxide.

13. The method of any one of embodiments 10-12, wherein the immunogenic composition comprises from about 0.25 mg to about 1.25 mg Al³⁺.

14. The method of any one of embodiments 10-12, wherein the immunogenic composition comprises about 0.50 mg, about 0.75 mg, or about 1.00 mg Al³⁺.

15. The method of embodiment 14, wherein the immunogenic compositions comprises about 0.75 mg Al³⁺.

16. The method of any one of embodiments 1-15, wherein the immunogenic composition does not comprise a saponin, a TLR4 agonist and/or a liposome.

17. The method of any one of embodiments 1-15, wherein the immunogenic composition does not comprise QS21, 3-O-deacylated monophosphoryl lipid A (3D-MPL1), dioleoyl phosphatidylcholine, and/or cholesterol.

18. The method of any one of embodiments 1-17, wherein the gE antigen and the oligonucleotide are present in the immunogenic composition at a ratio of from 1:15 to 1:120 (wt/wt), optionally wherein the gE antigen and the oligonucleotide are present in the immunogenic composition at a ratio of from 1:15 to 1:60 (wt/wt).

19. The method of any one of embodiments 1-17, wherein the gE antigen and the oligonucleotide are present in the immunogenic composition at a ratio of 1:15, 1:30, 1:60, 1:20, 1:40, 1:80, 1:30, 1:60, or 1:120 (wt/wt), optionally wherein the gE antigen and the oligonucleotide are present in the immunogenic composition at a ratio of from 1:30 (wt/wt), optionally wherein the gE antigen and the oligonucleotide are present in the immunogenic composition at a ratio of from 1:60 (wt/wt).

20. The method of any one of embodiments 13-19, wherein the oligonucleotide and the Al³⁺ are present in the immunogenic composition at a ratio of from 8:1 to 2:1 (wt/wt), optionally wherein the oligonucleotide and the Al³⁺ are present in the immunogenic composition at a ratio of 8:1 (wt/wt), optionally wherein the oligonucleotide and the Al³⁺ are present in the immunogenic composition at a ratio of 4:1 (wt/wt).

21. The method of any one of embodiments 1-20, wherein the human subject is 18 years of age or older.

22. The method of any one of embodiments 1-20, wherein the human subject is 50 years of age or older.

23. The method of embodiment 22, wherein the human subject is 50-69 years of age.

24. The method of any one of embodiments 1-23, wherein the human subject is suspected to have a latent VZV infection.

25. The method of any one of embodiments 1-24, wherein the human subject was not previously vaccinated against varicella or herpes zoster.

26. The method of any one of embodiments 1-25, wherein the human subject receives a first dose and a second dose of the immunogenic composition with the second dose administered from 1 month to 1 year after the first dose.

27. The method of embodiment 26, wherein the second dose is administered from 2 months to 6 months after the first dose, optionally wherein the second dose is administered about 2 months after the first dose.

28. The method of any one of embodiments 1-27, wherein the immunogenic composition is administered by intramuscular injection.

29. The method of any one of embodiments 1-28, wherein the increase in cell-mediated immunity against VZV comprises an increase in frequency of gE antigen-reactive, activated CD4+ T cells, wherein the activated CD4+ T cells express two or more activation markers selected from interleukin-2, interferon-gamma, tumor necrosis factor-alpha, and CD40L (CD154).

30. The method of any one of embodiments 1-29, wherein administration of the immunogenic composition results in an increase in humoral immunity against VZV in the subject relative to humoral immunity against VZV in the subject prior to administration of the immunogenic composition.

31. The method of embodiment 30, wherein the increase in humoral immunity against VZV comprises an increased concentration of gE antigen-reactive IgG.

32. The method of embodiment 30 or embodiment 31, wherein the increase in humoral immunity against VZV comprises an increase in concentration of gE- and/or VZV-neutralizing antibodies.

33. The method of any one of embodiments 30-32, wherein the increase in cell mediated immunity and the increase in humoral immunity are elicited in the subject by about two months after administration of the first dose of the immunogenic composition.

34. The method of any one of embodiments 30-32, wherein the increase in cell mediated immunity and the increase in humoral immunity are elicited in the subject by about one month after administration of the second dose of the immunogenic composition.

35. The method of any one of embodiments 30-34, wherein the increase in cell mediated immunity and the increase in humoral immunity are maintained in the subject for at least three months after administration of the second dose of the immunogenic composition.

36. The method of any one of embodiments 30-35, wherein administration of the immunogenic composition reduces risk of the subject developing herpes zoster relative to risk of a control subject who did not receive an immunogenic composition comprising the gE antigen.

37. The method of any one of embodiments 30-35, wherein administration of the immunogenic composition prevents the subject from developing herpes zoster, optionally for a period of at least 3 years after administration of the second dose of the immunogenic composition.

38. The method of any one of embodiments 30-35, wherein administration of the immunogenic composition prevents the subject from developing postherpetic neuralgia, optionally for a period of at least 3 years after administration of the second dose of the immunogenic composition.

39. The method of any one of embodiments 30-38, wherein the immunogenic composition is associated with a reduced risk of development of one or more solicited local reactions within 7 days following administration of the first dose and/or the second dose (day of vaccination and subsequent 6 days) relative to a comparator composition comprising the gE antigen and an AS01B adjuvant.

40. The method of embodiment 39, wherein the solicited local reactions comprise pain that prevents normal every day activities.

41. The method of any one of embodiments 30-40, wherein the immunogenic composition is associated with a reduced risk of development of one or more solicited systemic reactions within 7 days following administration of the first dose and/or the second dose (day of vaccination and subsequent 6 days) relative to a comparator composition comprising the gE antigen and an AS01B adjuvant.

42. The method of embodiment 41, wherein the solicited systemic reactions comprise fatigue, headache and/or myalgia that prevents normal every day activities, and/or fever above 39° C.

43. The method of any one of embodiments 30-42, wherein the immunogenic composition is associated with a reduced risk of development of Guillain-Barre syndrome within 42 days following administration of the first dose and/or the second dose (day of vaccination and subsequent 41 days) relative to a comparator composition comprising the gE antigen and an AS01B adjuvant.

44. The immunogenic composition for use in the method of any one of embodiments 1-43.

45. An immunogenic composition comprising: i) an unmethylated cytidine-phospho-guanosine (CpG)-containing oligonucleotide comprising the sequence of 5′-TGACTGTGAA CGTTCGAGAT GA-3′(SEQ ID NO: 1); ii) a truncated VZV glycoprotein E (gE) antigen; and iii) at least one excipient, wherein the gE antigen is a recombinant protein produced in mammalian cells and is present in the immunogenic composition in an amount of from about 25 μg to about 150 μg, the oligonucleotide is present in the immunogenic composition in an amount of from about 750 μg to about 6000 μg, and the at least one excipient comprises pharmaceutically acceptable buffer.

46. An immunogenic composition comprising i) an unmethylated cytidine-phospho-guanosine (CpG)-containing oligonucleotide comprising the sequence of 5′-TGACTGTGAA CGTTCGAGAT GA-3′(SEQ ID NO: 1); ii) a truncated VZV glycoprotein E (gE) antigen; and iii) at least one excipient, wherein the gE antigen is a recombinant protein produced in mammalian cells and is present in the immunogenic composition in an amount of about 100 μg, the oligonucleotide is present in the immunogenic composition in an amount of about 3000 μg or about 6000 μg, and the at least one excipient comprises pharmaceutically acceptable buffer.

47. The immunogenic composition of embodiment 45 or embodiment 46, further comprising an aluminum hydroxide adjuvant, wherein the immunogenic composition comprises from about 0.25 mg to about 1.25 mg Al³⁺.

48. The immunogenic composition of embodiment 47, wherein the immunogenic composition comprises about 0.75 mg Al³⁺.

49. The immunogenic composition of any one of embodiments 45-48, wherein the buffer is not a phosphate-containing buffer.

50. The immunogenic composition of any one of embodiments 45-48, wherein the buffer is a Tris buffer.

### EXAMPLES

Abbreviations: CMI (cell-mediated immunity); CpG (unmethylated cytidine-phospho-guanosine); CTRL (control); gE (glycoprotein E); ELISA (enzyme-linked immunosorbent assay); GMC (geometric mean concentration); GMFI (geometric mean fold increase); HZ (herpes zoster); IFNγ (interferon-gamma); IL-2 (interleukin-2); IM (intramuscular); mcg or μg (microgram); mcl or μl (microliter); PBMC (peripheral blood mononuclear cell); TLR9 (toll-like receptor 9); TNFα or TNF (tumor necrosis factor-alpha); VRR (vaccine response rate); and VZV (varicella zoster virus).

### Example 1

### Safety, Tolerability, and Immunogenicity of CpG-Adjuvanted Recombinant Zoster Vaccine Compared to an AS01B-Adjuvanted, Recombinant Zoster Vaccine in Human Subjects

This example provides a description of a phase I clinical trial conducted in healthy adults to compare effects of administration of two doses of Z-1018 (vaccine comprising recombinant VZV gE adjuvanted with CpG 1018® adjuvant, Dynavax Technologies Corporation, Emeryville, CA) over the course of two months to administration of two doses of SHINGRIX® (vaccine comprising VZV gE adjuvanted with AS01B, GlaxoSmithKline, Research Triangle Park, NC) administered over the course of two months.

Antigen: Recombinant VZV glycoprotein E (gE) was obtained by cell culture of genetically engineered Chinese Hamster Ovary host cells expressing a truncated version of gE lacking the transmembrane anchor and carboxy-terminal domain, and is therefore secreted into the supernatant. The cell culture was supported by media containing amino acids, but no albumin, antibiotics or animal derived proteins. gE was purified by chromatography and lyophilized for future use as described (Haumont et al., Virus Res, 40:199-204, 1996). For the phase I clinical trial, VZV gE was obtained commercially.

Primary Objective: To assess the safety and tolerability of Z-1018. Endpoints include assessment of adverse events: solicited local and systemic post-injection reactions; adverse events; and serious adverse events.

Secondary Objective: To assess the immunogenicity of Z-1018 compared with SHINGRIX® at 4 weeks after second injection. Endpoints include determination of: 1) numbers of CD4+ T cells expressing at least two of the following activation markers: IFNγ, IL-2, TNFα, and CD40L; 2) geometric mean concentration (GMC) of IgG antibodies to VZV gE antigen; and 3) vaccine response rate (VRR). A desirable endpoint is an increase in CD4+ T cells reactive with gE in PBMC, which have a 2-fold or greater increase over baseline in expression of 2 or more of IFNγ, IL-2, TNFα, and CD40L.

Exploratory Objective: To further assess immunogenicity of Z-1018. Endpoints include assessment of: 1) levels of gE neutralizing antibodies; 2) T cell phenotype; 3) B cell phenotype; and 4) peripheral blood mononuclear cell (PBMC) gene expression profile.

Study Design. This study was conducted as a randomized, active-controlled, dose-escalation multi-center study of 2 doses (Day 1 and Week 8) of an investigational herpes zoster (HZ) vaccine (Z-1018), combining herpes zoster gE antigen with an oligodeoxynucleotide, Toll-like receptor 9 (TLR9) agonist adjuvant (CpG 1018@adjuvant) with and without alum in healthy volunteers aged 50 to 69 years. Subjects were randomized to receive escalating dose levels of Z-1018 or SHINGRIX® as follows:
i) Z-1018 Dose Level 1: 100 mcg gE+3000 mcg CpG 1018@adjuvant;
ii) Z-1018 Dose Level 1a: 100 mcg gE+3000 mcg CpG 1018@adjuvant+alum;
iii) Z-1018 Dose Level 2: 100 mcg gE+6000 mcg CpG 1018® adjuvant;
iv) Z-1018 Dose Level 2a: 100 mcg gE+6000 mcg CpG 1018@adjuvant+alum: or
v) SHINGRIX®.

All subjects received 2 doses of their assigned treatment and underwent assessments of safety and immunogenicity. Subjects were followed for safety for 12 weeks after the last study injection (i.e., 12 weeks after second injection at week 8=20 weeks after first injection). All subjects had a pre-vaccination blood draw and received the assigned treatment vaccine at Day 1.

Blood draws for measuring post-vaccination antibody levels and exploratory evaluations were done at Weeks 8, 12, and 20. Subjects received the second dose of their assigned treatment at Week 8 after the blood draws. Safety assessments were done at Baseline (Day 1), Day 8, Week 4, Week 12, Week 16, and Week 20. Duration of subject participation in the Screening and Treatment parts of the study was −24 weeks.

Study Population. Healthy male and female subjects of 50-69 years of age were selected. Exclusion criteria included any one of the following criteria: seropositive for human immunodeficiency virus; history of herpes zoster; previous vaccination against varicella or herpes zoster; has received any non-live vaccine within 14 days of the first injection; has received any live vaccine, a systemic corticosteroids (inhaled steroids are permissible), an immunomodulator, or an immune suppressive medication, granulocyte-stimulating factor, or granulocyte-macrophage colony stimulating factor within 28 days of the first injection; is undergoing or expects to receive chemotherapy; has a diagnosis of cancer within last 5 years; and a history of autoimmune disease.

Study Treatments. The Investigational Medicinal Product (Z-1018) or the Active Comparator (SHINGRIX®) were administered as a single intramuscular (IM) injection into the deltoid muscle on day 1 and week 8.

A 1 ml dose of Z-1018 contains 100 mcg gE, combined with CpG 1018® adjuvant (Dynavax Technologies Corporation. Emeryville, CA) at a dose of 3000 mcg or 6000 mcg (without or with aluminum hydroxide [alum] at a dose of 750 mcg Al³⁺). CpG 1018® adjuvant is a single-stranded, 22-base phosphorothioate 2′-deoxyribo-oligonucleotide prepared by standard solid phase chemistry techniques (5′-TGACTGTGAA CGTTCGAGAT GA-3′, set forth as SEQ ID NO:1). CpG 1018@adjuvant has a molecular mass of approximately 7150 Daltons. Alum in Z-1018 is ALHYDROGEL® vaccine adjuvant marketed by Croda International Plc (East Yorkshire, UK).

A 0.5 ml dose of SHINGRIX® contains 50 mcg gE, combined with AS01B adjuvant (liposomal formulation containing 1 mg dioleoyl phosphatidylcholine [DOPC], 250 mcg cholesterol, 50 mcg 3-O-desacyl-4′-monophosphoryl lipid A [MPL] from Salmonella minnesota, and 50 mcg QS-21 saponin from Quillaja saponaria Molina, fraction 21).

Immunogenicity Assessments. IgG antibodies to gE (anti-gE), and T and B cell responses to gE were assessed at baseline (Day 1), Week 8, Week 12, and Week 20. Neutralizing antibodies to gE were also assessed at baseline (Day 1), Week 8, Week 12, and Week 20.

The Safety Population comprises all subjects who received at least 1 dose of the study vaccine, excluding subjects who have no on-study data.

The Per-protocol (PP) Population for immunogenicity analyses comprises all subjects who received the two doses of study vaccine, have no major protocol violations (to be specified in the statistical analysis plan), and have CD4+ T cells obtained within study visit windows at Week 12.

The modified intent-to-treat (mITT) population for the immunogenicity analysis comprises all eligible subjects who received at least one dose of study vaccine and have a post-injection immunogenicity evaluation.

For immunogenicity analyses, the response rates for IgG and neutralizing antibodies to gE were evaluated. For cell-mediated immune (CMI) responses, frequencies of CD4+ T cells expressing ≥2 activation markers were measured. Additionally, the geometric mean concentrations (GMCs) were evaluated for antibodies to gE at Weeks 8 and 12. Geometric mean ratio (GMR) (e.g., Week 8 vs Day 1, Week 12 vs Day 1) was evaluated for antibodies to gE by dose level. T and B cell phenotypes and safety data are analyzed descriptively.

VZV Glycoprotein E (gE) ELISA. Wells of a flat bottom microplate were coated overnight with 0.1 mcg VZV gE antigen, washed, blocked in a buffer containing 5% bovine serum albumin, and re-washed. Standards, controls and human serum samples (diluted 1:50 or higher) were added in duplicate to wells of the microplate, which was incubated for 1.0-1.5 hours at room temperature. The standard used was an anti-VZV immunoglobulin World Health Organization international standard (National Institute for Biological Standards and Control, Catalog No. W1044). After washing, the detection antibody (diluted 1:50,000) was added to all wells of the microplate, which was incubated for 1.0-1.25 hours at room temperature. The detection antibody used was a mouse anti-human IgG-peroxidase (Jackson ImmunoResearch, Catalog No. 209-035-98). After washing, the 3,3′,5,5′-tetramethylbenzidine substrate (Life Technologies, Catalog No. 002023) was added to all wells of the microplate, and allowed to react for about 10 minutes before color development was stopped by addition of a 1N sulfuric acid stop solution. Absorbance was measured and concentration determined using a Tecan SPARK Microplate Reader and Magellan Data Analysis Software.

Intracellular Staining Assay. PBMC were isolated from blood samples obtained from study subjects and cryopreserved. Frozen PBMC were gently thawed, washed in complete medium containing benzonase, and transferred to a 6-well cell culture plate in complete medium without benzonase. Cells were rested overnight in an incubator at 5% CO₂, 37° C. Rested cells were washed, counted, and resuspended in fresh complete medium. When cell viability was at least 60-70%, cells were distributed to wells of a 96-well cell culture plate and stimulated in complete medium containing: DMSO (0.8%), a series of peptides (15 mers with 11 aa overlaps) spanning the VZV gEprotein (1 mcg/well). After cells were stimulated for 2 hours in an incubator at 5% CO₂, 37° C., an inhibitor mix containing Monensin and Brefeldin A was added to each well, and the cell culture plate was returned to the incubator for another 16-18 hours. Cells were then washed, resuspended in a live/dead viability dye and incubated in the dark for 20 minutes at room temperature. After washing, cells were resuspended in cold Cytofix-Cytoperm and incubated in the dark for 20 minutes at 2-8° C. Cells were washed again and resuspended in Perm/Wash Buffer. Pelleted cells were resuspended in an intracellular staining panel mix including fluorochrome-labeled antibodies against CD3, CD4, CD8, IFNγ, IL-2, TNFα, and CD40L and incubated for 20 minutes in the dark at room temperature. Stained cells were washed and resuspended in a stain buffer and stored at 2-8° C. until analysis using an Attune NxT Flow Cytometer.

### Results—Safety and Tolerability

Post-injection reactions and adverse events were assessed in the safety population, which comprised all subjects who received at least one dose of a study vaccine. Characteristics of the safety population are shown in Table 1. More women than men were present in the Z-1018 groups, while the Shingrix® group was more gender balanced. The mean age was similar across all study groups.

TABLE 1
Gender and Age of Safety Population

| | | Z-1018 | | Z-1018 | |
|---|---|---|---|---|---|
| | Z-1018 | 3 mg + | Z-1018 | 6 mg + | |
| | 3 mg | alum | 6 mg | alum | Shingrix ® |
| | (N = 30) | (N = 29) | (N = 31) | (N = 30) | (N = 30) |
| Sex | | | | | |
| Male | 11 (36.7%) | 13 (44.8%) | 11 (35.5%) | 9 (30.0%) | 14 (46.7%) |
| Female | 19 (63.3%) | 16 (55.2%) | 20 (64.5%) | 21 (70.0%) | 16 (53.3%) |
| Age (years) | | | | | |
| n | 30 | 29 | 31 | 30 | 30 |
| Mean (SD) | 59.3 (5.70) | 60.0 (6.37) | 57.1 (5.29) | 59.7 (4.94) | 60.1 (4.43) |
| Median | 59.5 | 60.0 | 56.0 | 60.5 | 59.5 |
| Min, Max | 50.0, 68.0 | 51.0, 69.0 | 50.0, 69.0 | 51.0, 68.0 | 51.0, 68.0 |

Overall rates of reactogenicity are shown in Table 2. Local and systemic reactogenicity rates of the safety population are shown in Table 3 and Table 4, respectively.

TABLE 2
Overview of Reactogenicity of Safety Population\hat{ }

| | Z-1018 | Z-1018 | Z-1018 | Z-1018 | |
|---|---|---|---|---|---|
| | 3 mg | 3 mg + alum | 6 mg | 6 mg + alum | Shingrix ® |
| | (N = 30) | (N = 29) | (N = 31) | (N = 30) | (N = 30) |
| Any Reactions | | | | | |
| Reactions n (%) | 28 (93.3%) | 27 (93.1%) | 27 (87.1%) | 24 (80.0%) | 30 (100.0%) |
| Severe Reactions n (%) | 1 (3.3%) | 1 (3.4%) | 2 (6.5%) | 4 (13.3%) | 6 (20.0%) |
| Local Reactions | | | | | |
| Reactions n (%) | 26 (86.7%) | 26 (89.7%) | 25 (80.6%) | 23 (76.7%) | 29 (96.7%) |
| Severe Reactions n (%) | 0 | 0 | 1 (3.2%) | 2 (6.7%) | 3 (10.0%) |
| Systemic Reactions | | | | | |
| Reactions n (%) | 21 (70.0%) | 21 (72.4%) | 22 (71.0%) | 20 (66.7%) | 30 (100.0%) |
| Severe Reactions n (%) | 1 (3.3%) | 1 (3.4%) | 1 (3.2%) | 2 (6.7%) | 5 (16.7%) |

\hat{ }Redness/Swelling: Greater than or equal to 25 mm, Severe: Greater than 100 mm.
Fever: Greater than or equal to 38° C., Severe: 39° C. to 40° C., Potentially Life Threatening: >40° C.

TABLE 3
Overview of Local Reactogenicity of Safety Population

| | | Z-1018 | | Z-1018 | |
|---|---|---|---|---|---|
| | Z-1018 | 3 mg + | Z-1018 | 6 mg + | |
| | 3 mg | alum | 6 mg | alum | Shingrix ® |
| | (N = 30) | (N = 29) | (N = 31) | (N = 30) | (N = 30) |
| Local | | | | | |
| Reactions | | | | | |
| Total | 26 (86.7%) | 26 (89.7%) | 25 (80.6%) | 23 (76.7%) | 29 (96.7%) |
| Severe | 0 | 0 | 1 (3.2%) | 2 (6.7%) | 3 (10.0%) |
| Injection | | | | | |
| Site Redness | | | | | |
| Total | 2 (6.7%) | 1 (3.4%) | 3 (9.7%) | 2 (6.7%) | 9 (30.0%) |
| Severe | 0 | 0 | 1 (3.2%) | 1 (3.3%) | 3 (10.0%) |
| Injection | | | | | |
| Site Swelling | | | | | |
| Total | 3 (10.0%) | 1 (3.4%) | 1 (3.2%) | 4 (13.3%) | 7 (23.3%) |
| Severe | 0 | 0 | 0 | 2 (6.7%) | 2 (6.7%) |
| Injection | | | | | |
| Site Pain | | | | | |
| Total | 26 (86.7%) | 25 (86.2%) | 25 (80.6%) | 23 (76.7%) | 29 (96.7%) |
| Severe | 0 | 0 | 0 | 0 | 0 |
| Pruritis | | | | | |
| Total | 6 (20.0%) | 3 (10.3%) | 3 (9.7%) | 3 (10.0%) | 10 (33.3%) |
| Severe | 0 | 0 | 0 | 0 | 0 |

\hat{ }Redness/Swelling: Greater than or equal to 25 mm, Severe: Greater than 100 mm.

TABLE 4
Overview of Systemic Reactogenicity of Safety Population

| | | Z-1018 | | Z-1018 | |
|---|---|---|---|---|---|
| | Z-1018 | 3 mg + | Z-1018 | 6 mg + | |
| | 3 mg | alum | 6 mg | alum | Shingrix ® |
| | (N = 30) | (N = 29) | (N = 31) | (N = 30) | (N = 30) |
| Systemic | | | | | |
| Reactions | | | | | |
| Total | 21 (70.0%) | 21 (72.4%) | 22 (71.0%) | 20 (66.7%) | 30 (100.0%) |
| Severe | 1 (3.3%) | 1 (3.4%) | 1 (3.2%) | 2 (6.7%) | 5 (16.7%) |
| Fever [b] | | | | | |
| Total | 0 | 0 | 0 | 1 (3.3%) | 0 |
| Severe | 0 | 0 | 0 | 0 | 0 |
| Malaise | | | | | |
| Total | 13 (43.3%) | 12 (41.4%) | 13 (41.9%) | 12 (40.0%) | 21 (70.0%) |
| Severe | 0 | 0 | 0 | 0 | 0 |
| Headache | | | | | |
| Total | 11 (36.7%) | 15 (51.7%) | 16 (51.6%) | 13 (43.3%) | 21 (70.0%) |
| Severe | 0 | 1 (3.4%) | 0 | 0 | 0 |
| Myalgia | | | | | |
| Total | 10 (33.3%) | 7 (24.1%) | 13 (41.9%) | 9 (30.0%) | 19 (63.3%) |
| Severe | 0 | 0 | 1 (3.2%) | 1 (3.3%) | 4 (13.3%) |
| Fatigue | | | | | |
| Total | 13 (43.3%) | 16 (55.2%) | 17 (54.8%) | 16 (53.3%) | 27 (90.0%) |
| Severe | 1 (3.3%) | 1 (3.4%) | 0 | 1 (3.3%) | 3 (10.0%) |
| Nausea | | | | | |
| Total | 7 (23.3%) | 4 (13.8%) | 6 (19.4%) | 3 (10.0%) | 9 (30.0%) |
| Severe | 0 | 0 | 0 | 0 | 0 |
| Vomiting | | | | | |
| Total | 1 (3.3%) | 0 | 1 (3.2%) | 0 | 0 |
| Severe | 0 | 0 | 0 | 0 | 0 |
| Diarrhea | | | | | |
| Total | 3 (10.0%) | 5 (17.2%) | 6 (19.4%) | 4 (13.3%) | 8 (26.7%) |
| Severe | 0 | 0 | 0 | 1 (3.3%) | 0 |
| Chills | | | | | |
| Total | 7 (23.3%) | 5 (17.2%) | 6 (19.4%) | 4 (13.3%) | 15 (50.0%) |
| Severe | 0 | 0 | 0 | 1 (3.3%) | 0 |

In this study, most participants in the Z-1018 groups reported some reactogenicity, but most reactions were mild or moderate. Injection site pain was the most common local reaction. Fatigue was the most commonly reported systemic reaction, while nausea and diarrhea were the most frequently reported gastrointestinal symptom. While there was no clear dose response in the reporting rate of local or systemic reactions in the Z-1018 groups, more severe reactions were reported in the Z-1018 6 mg+alum group than the other three Z-1018 groups.

The reporting rates of any, local and systemic reactions were higher following Shingrix® than Z-1018. In addition, the number of reports of moderate or severe reactions were greater following Shingrix® than any formulation of Z-1018. Observations from the safety population indicate that all formulations of Z-1018 have a better tolerability profile than Shingrix®.

### Results—Immunogenicity

VZV gE-reactive humoral and cellular immune response were assessed in the per protocol population, which comprised all subjects who received two doses of a study vaccine, had no major protocol violations, and had CD4+ T cells obtained within the study visit window 4 weeks after the second injection. Characteristics of the per protocol population are shown in Table 5. All Z-1018 groups had comparatively more women than did the Shingrix® group, while age was well balanced among all groups.

TABLE 5
Gender and Age of Per Protocol Population

| | | Z-1018 | | Z-1018 | |
|---|---|---|---|---|---|
| | Z-1018 | 3 mg + | Z-1018 | 6 mg + | |
| | 3 mg | alum | 6 mg | alum | Shingrix ® |
| | (N = 21) | (N = 23) | (N = 25) | (N = 24) | (N = 23) |
| Sex | | | | | |
| Male | 8 (38.1%) | 10 (43.5%) | 9 (36.0%) | 7 (29.2%) | 12 (52.2%) |
| Female | 13 (61.9%) | 13 (56.5%) | 16 (64.0%) | 17 (70.8%) | 11 (47.8%) |
| Age (years) | | | | | |
| n | 21 | 23 | 25 | 24 | 23 |
| Mean (SD) | 59.8 (5.74) | 61.0 (6.49) | 57.1 (4.84) | 59.7 (4.54) | 60.1 (4.68) |
| Median | 59.0 | 62.0 | 56.0 | 61.0 | 60.0 |
| Min, Max | 50.0, 68.0 | 51.0, 69.0 | 50.0, 69.0 | 51.0, 66.0 | 51.0, 68.0 |

Levels of anti-gE IgG antibodies (IU/mL) are shown as geometric mean concentration (GMC) in Table 6, as geometric mean fold increase (GMFI) in Table 7, and as vaccine response rate in Table 8.

TABLE 6
Anti-gE IgG Antibodies Shown as GMC (95% CI)

| | | Z-1018 | | Z-1018 | |
|---|---|---|---|---|---|
| | Z-1018 | 3 mg + | Z-1018 | 6 mg + | |
| | 3 mg | alum | 6 mg | alum | Shingrix ® |
| | (N = 21) | (N = 23) | (N = 25) | (N = 24) | (N = 23) |
| Visit | | | | | |
| Day 1 | 1.4 | 1.1 | 1.1 | 1.2 | 1.7 |
| | (1.0-2.0) | (0.8-1.6) | (0.6-2.0) | (0.8-2.0) | (1.1-2.6) |
| Week 8 | 22.6 | 22.5 | 19.5 | 29.6 | 27.0 |
| | (14.5-35.3) | (13.5-37.4) | (13.2-28.6) | (19.5-45.0) | (19.4-37.5) |
| Week 12 | 49.2 | 46.1 | 49.3 | 51.4 | 63.1 |
| | (39.4-61.3) | (32.3-65.8) | (39.1-62.1) | (38.7-68.1) | (51.6-77.1) |

TABLE 7
Anti-gE IgG Antibodies Shown as GMFI (95% CI)

| | | Z-1018 | | Z-1018 | |
|---|---|---|---|---|---|
| | Z-1018 | 3 mg + | Z-1018 | 6 mg + | |
| | 3 mg | alum | 6 mg | alum | Shingrix ® |
| | (N = 21) | (N = 23) | (N = 25) | (N = 24) | (N = 23) |
| Visit | | | | | |
| Week 8/ | 16.2 | 20.0 | 17.1 | 24.3 | 15.8 |
| Day 1 | (10.1-25.9) | (10.7-37.3) | (10.0-29.5) | (14.7-40.0) | (10.0-24.9) |
| Week 12/ | 35.2 | 41.0 | 43.3 | 42.1 | 36.9 |
| Day 1 | (24.1-51.5) | (24.9-67.7) | (24.9-75.3) | (26.1-68.0) | (24.9-54.8) |

TABLE 8
Anti-gE IgG Antibodies Shown as Vaccine Response Rate

| | | Z-1018 | | Z-1018 | |
|---|---|---|---|---|---|
| | Z-1018 | 3 mg + | Z-1018 | 6 mg + | |
| | 3 mg | alum | 6 mg | alum | Shingrix ® |
| | (N = 21) | (N = 23) | (N = 25) | (N = 24) | (N = 23) |
| | % | % | % | % | % |
| Visit | (95% CI) | (95% CI) | (95% CI) | (95% CI) | (95% CI) |
| Week 8 | 90.5 | 87.0 | 88.0 | 95.8 | 95.7 |
| | (69.6-98.8) | (66.4-97.2) | (68.8-97.5) | (78.9-99.9) | (78.1-99.9) |
| Week 12 | 100.0 | 95.7 | 100.0 | 95.8 | 100.0 |
| | (83.9-100.0) | (78.1-99.9) | (86.3-100.0) | (78.9-99.9) | (85.2-100.0) |

\hat{ }Vaccine response was defined as an at least 4-fold increase in GMC post-vaccination to pre-vaccination (Week 8 Antibody Level/Day 1 Antibody Level and Week 12 Antibody Level/Day 1 Antibody Level).
The confidence intervals were calculated using the two-sided Clopper-Pearson method.

The GMCs achieved at 12 weeks were comparable across all four Z-1018 groups. The GMCs in the Z-1018 groups were lower than in the Shingrix® group. However, conclusions across groups are difficult to draw because of the higher baseline GMC in the Shingrix® group. At 12 weeks, the GMFIs of all Z-1018 groups were comparable or higher than Shingrix®. Among the Z-1018 groups, the highest GMFIs were observed in the Z-1018 6 mg groups with and without alum. A high VRR was achieved in all Z-1018 groups at week 12, and was comparable to Shingrix®.

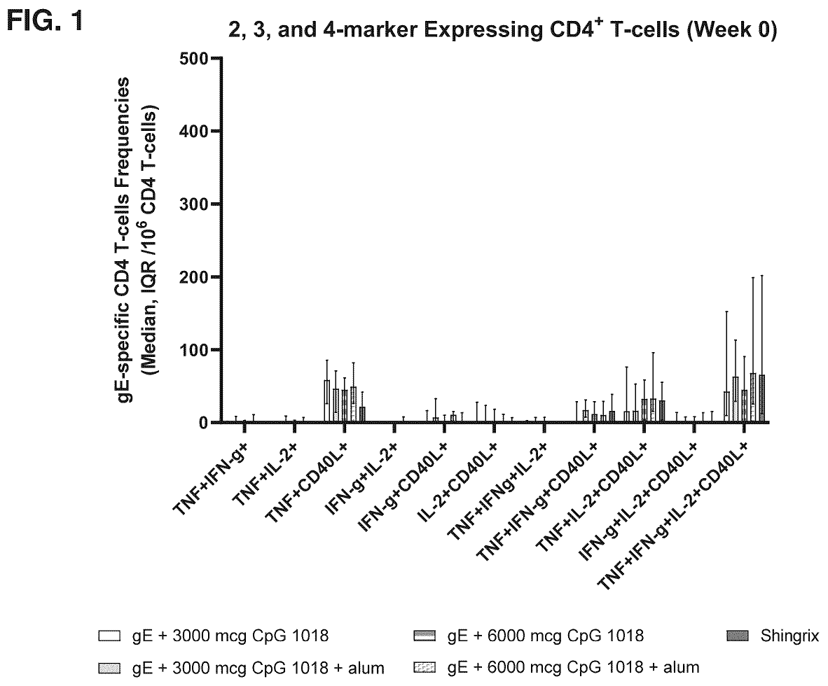

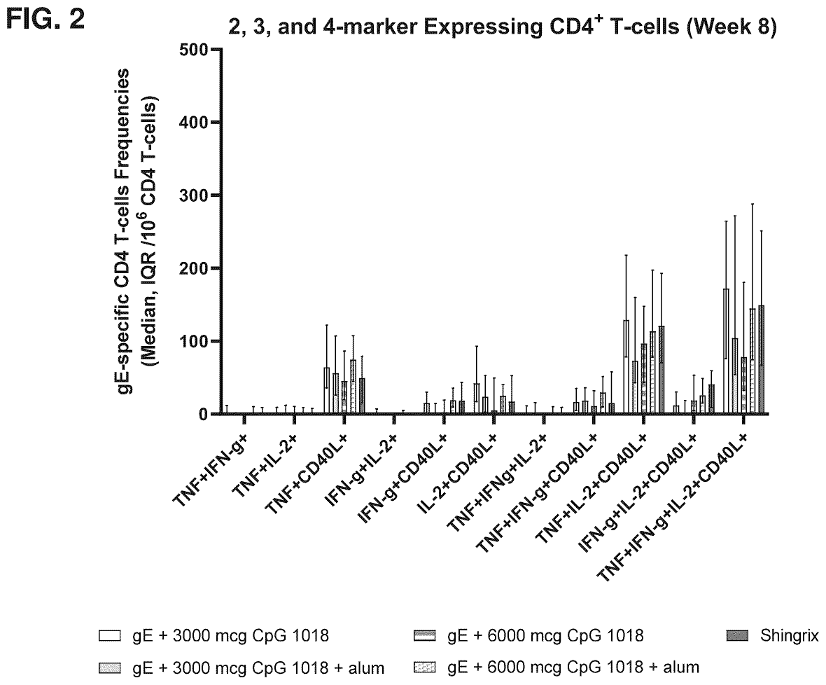

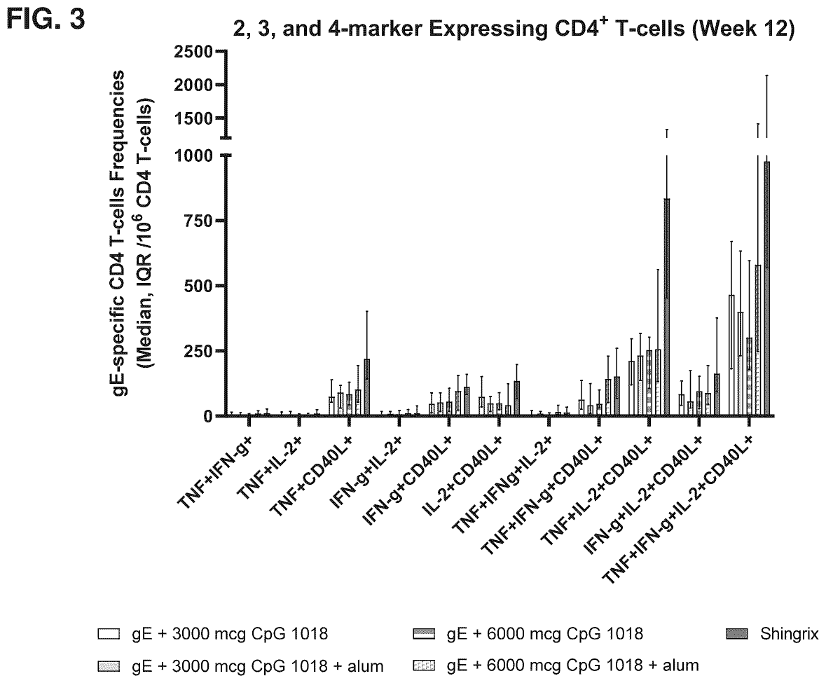

Levels of gE-reactive CD4+ T cells expressing two or more activation markers (referred to herein as “CD4+(2+) T cells”) are shown as median frequency per 10⁶ CD4+ T cells in Table 9 and FIG. 1-3, as geometric mean fold increase (GMFI) in Table 10, and as a percentage of subjects with a 2-fold increase post-vaccination to pre-vaccination in Table 11.

TABLE 9
Descriptive Statistics of Frequencies
of gE-reactive CD4+(2+) T cells

| | | Z-1018 | | Z-1018 | |
|---|---|---|---|---|---|
| | Z-1018 | 3 mg + | Z-1018 | 6 mg + | |
| | 3 mg | alum | 6 mg | alum | Shingrix ® |
| | (N = 21) | (N = 23) | (N = 25) | (N = 24) | (N = 23) |
| Week 0 | | | | | |
| n | 20 | 23 | 25 | 23 | 21 |
| Median | 144.5 | 180.0 | 133.0 | 187.0 | 151.0 |
| Min, Max | 2.0, | 7.0, | 2.0, | 28.0, | 2.0, |
| | 450.0 | 638.0 | 1020.0 | 804.0 | 703.9 |
| Q1 | 71.6 | 68.0 | 52.0 | 93.0 | 32.0 |
| Q3 | 295.5 | 249.0 | 160.0 | 345.0 | 311.0 |
| Week 8 | | | | | |
| n | 21 | 23 | 25 | 24 | 22 |
| Median | 461.0 | 330.0 | 258.0 | 454.5 | 467.0 |
| Min, Max | 189.0, | 2.0, | 2.0, | 155.0, | 55.1, |
| | 1972.0 | 1531.0 | 1166.0 | 1375.0 | 1652.0 |
| Q1 | 375.4 | 223.6 | 191.0 | 279.0 | 318.0 |
| Q3 | 685.7 | 567.0 | 477.5 | 767.0 | 555.0 |
| Week 12 | | | | | |
| n | 21 | 23 | 25 | 24 | 22 |
| Median | 1087.4 | 1002.0 | 1058.0 | 1369.5 | 2893.5 |
| Min, Max | 176.9, | 296.0, | 2.0, | 363.0, | 955.0, |
| | 2659.0 | 5809.0 | 5778.0 | 5562.0 | 11553.0 |
| Q1 | 725.0 | 682.0 | 695.0 | 782.5 | 1790.0 |
| Q3 | 1789.0 | 1304.0 | 1427.0 | 3143.0 | 4864.5 |

TABLE 10
gE-reactive CD4+(2+) T cells Shown as GMFI (95% CI)

| | | Z-1018 | | Z-1018 | |
|---|---|---|---|---|---|
| | Z-1018 | 3 mg + | Z-1018 | 6 mg + | |
| | 3 mg | alum | 6 mg | alum | Shingrix ® |
| | (N = 20) | (N = 23) | (N = 25) | (N = 23) | (N = 21) |
| Visit | | | | | |
| Week 8/ | 4.6 | 2.0 | 2.7 | 2.6 | 5.0 |
| Day 1 | (2.3-9.2) | (1.2-3.4) | (1.1-6.7) | (1.9-3.7) | (2.3-11.0) |
| Week 12/ | 9.6 | 7.2 | 10.0 | 8.7 | 35.6 |
| Day 1 | (4.5-20.8) | (4.8-10.8) | (3.9-25.3) | (5.7-13.2) | (15.1-84.0) |

TABLE 11
Percentages of Subjects with Two-Fold Increase in gE-reactive CD4+(2+) T cells\hat{ }

| | Z-1018 3 mg | | Z-1018 3 mg + alum | | Z-1018 6 mg | | Z-1018 6 mg + alum | | Shingrix ® | |
|---|---|---|---|---|---|---|---|---|---|---|
| | | % | | % | | % | | % | | % |
| Visit | n/N | (95% CI) | n/N | (95% CI) | n/N | (95% CI) | n/N | (95% CI) | n/N | (95% CI) |
| Week 8 | 15/21 | 71.43 | 14/23 | 60.87 | 13/25 | 52.00 | 15/24 | 62.50 | 16/22 | 72.73 |
| | | (47.82-88.72) | | (38.54-80.29) | | (31.31-72.20) | | (40.59-81.20) | | (49.78-89.27) |
| Week 12 | 19/21 | 90.48 | 22/23 | 95.65 | 23/25 | 92.00 | 21/24 | 87.50 | 21/22 | 95.45 |
| | | (69.62-98.83) | | (78.05-99.89) | | (73.97-99.02) | | (67.64-97.34) | | (77.16-99.88) |

\hat{ }N = number of evaluable subjects; CI = confidence interval.
n = number of subjects with 2-fold increase post-vaccination to pre-vaccination.
(Week 8 CD4+(2+) T cells/Day 1 CD4+(2+) T cells; and Week 12 CD4+(2+) T cells/Day 1 CD4+(2+) T cells).
The confidence intervals on vaccine response rate was calculated using the two-sided Clopper-Pearson method.

The frequency of gE-reactive CD4+(2+) T cells expressing different combinations of activation markers (two or more of IFNγ, IL-2, TNFα, and CD40L) were similar across all groups at Week 8. However, frequency of gE-reactive CD4+(2+) T cells was greater for Shingrix® than the four Z-1018 groups at Week 12. For Z-1018 groups, as well as Shingrix®, gE-reactive CD4+ T cells expressing all four activation markers occurred at the highest frequency. Robust GMFIs in CD4+(2+) T cell frequencies at Week 12 over baseline were observed in all four Z-1018 groups. At Week 12, high vaccine response rates based on CD4+(2+) T cell frequencies were seen in each Z-1018 group, and were comparable to Shingrix®. A summary of the immunogenicity data for the four Z-1018 groups is shown in Table 12.

TABLE 12
Immunogenicity Data for CpG 1018 ® Adjuvant (Z-1018) Groups

| | | | Z-1018 | | Z-1018 |
|---|---|---|---|---|---|
| | | Z-1018 | 3 mg + | Z-1018 | 6 mg + |
| | Measure | 3 mg | alum | 6 mg | alum |
| Anti-gE | GMFI | 35.2 | 41.0 | 43.3 | 42.1 |
| IgG | VRR | 100 | 95.7 | 100 | 95.8 |
| gE-reactive | GMFI | 9.6 | 7.2 | 10.0 | 8.7 |
| CD4+(2+) | VRR | 90.5 | 95.7 | 92.0 | 97.5 |
| T cells | | | | | |

Although the foregoing disclosure has been described in some detail by way of illustration and example for purposes of clarity and understanding, it will be apparent to those skilled in the art that certain changes and modifications may be practiced. Therefore, the examples should not be construed as limiting the scope of the disclosure, which is delineated by the appended claims.

What is claimed:

1. A method of increasing cell-mediated immunity against varicella zoster virus (VZV) in a human subject in need thereof, the method comprising:
administering to the human subject an immunogenic composition comprising: i) an unmethylated cytidine-phospho-guanosine (CpG)-containing oligonucleotide comprising the sequence of 5′-TGACTGTGAA CGTTCGAGAT GA-3′(SEQ ID NO: 1); and ii) a truncated VZV glycoprotein E (gE) antigen, which are present in the immunogenic composition in amounts effective to increase cell-mediated immunity against VZV relative to cell-mediated immunity against VZV in the subject prior to administration of the immunogenic composition, wherein a first dose and a second dose of the immunogenic composition is administered to the human subject, with the second dose administered from 2 months to 6 months after the first dose.

2. The method of claim 1, wherein the immunogenic composition comprises: from about 25 μg to about 150 μg of the gE antigen, and from about 750 μg to about 6000 μg of the oligonucleotide.

3. The method of claim 1, wherein the immunogenic composition comprises from about 50 μg to about 100 μg of the gE antigen, and from about 1500 μg to about 6000 μg of the oligonucleotide.

4. The method of claim 1, wherein the immunogenic composition comprises about 25 μg, about 50 μg, about 75 μg, about 100 μg, about 125 μg, or about 150 μg of the gE antigen, and about 750 μg, about 1500 μg, about 3000 μg, or about 6000 μg of the oligonucleotide.

5. The method of claim 4, wherein the immunogenic composition comprises about 100 μg of the gE antigen, and about 3000 μg or 6000 μg of the oligonucleotide.

6. A method of increasing an immune response against varicella zoster virus (VZV) in a human subject in need thereof, the method comprising:
administering to the human subject an immunogenic composition comprising: i) an unmethylated cytidine-phospho-guanosine (CpG)-containing oligonucleotide comprising the sequence of 5′-TGACTGTGAA CGTTCGAGAT GA-3′(SEQ ID NO: 1); and ii) a truncated VZV glycoprotein E (gE) antigen, wherein the immunogenic composition comprises about 100 μg of the gE antigen and about 3000 μg or about 6000 μg of the oligonucleotide.

7. The method of claim 5 or claim 6, wherein the immunogenic composition comprises about 100 μg of the gE antigen, and about 3000 μg of the oligonucleotide.

8. The method of claim 5 or claim 6, wherein the immunogenic composition comprises about 100 μg of the gE antigen, and about 6000 μg of the oligonucleotide.

9. The method of any one of claims 1-8, wherein the oligonucleotide comprises at least one phosphorothioate linkage, or wherein all nucleotide linkages are phosphorothioate linkages.

10. The method of any one of claims 1-9, wherein the oligonucleotide is a single-stranded oligodeoxynucleotide.

11. The method of any one of claims 1-10, wherein the gE antigen is a recombinant protein devoid of transmembrane and intravirion domains of a full-length VZV gE antigen.

12. The method of claim 11, wherein the gE antigen comprises the amino acid sequence of SEQ ID NO:4, or an amino acid sequence that is at least 90%, 95%, 96%, 97%, 98%, or 99% identical to SEQ ID NO:4.

13. The method of claim 11, wherein the gE antigen comprises the amino acid sequence of SEQ ID NO:5, or an amino acid sequence that is at least 90%, 95%, 96%, 97%, 98%, or 99% identical to SEQ ID NO:5.

14. The method of any one of claims 11-13, wherein the recombinant protein is produced in mammalian cells.

15. The method of any one of claims 1-14, wherein the immunogenic composition further comprises an aluminum salt adjuvant.

16. The method of claim 15, wherein the aluminum salt adjuvant comprises one or more of the group consisting of amorphous aluminum hydroxyphosphate sulfate, aluminum hydroxide, aluminum phosphate, and potassium aluminum sulfate.

17. The method of claim 15, wherein the aluminum salt adjuvant comprises aluminum hydroxide.

18. The method of any one of claims 15-17, wherein the immunogenic composition comprises from about 0.25 mg to about 1.25 mg Al³⁺.

19. The method of any one of claims 15-17, wherein the immunogenic composition comprises about 0.50 mg, about 0.75 mg, or about 1.00 mg Al³⁺.

20. The method of claim 19, wherein the immunogenic composition comprises about 0.75 mg Al³⁺.

21. The method of any one of claims 15-20, wherein the immunogenic composition further comprises a pharmaceutically acceptable buffer.

22. The method of claim 21, wherein the buffer is not a phosphate-containing buffer.

23. The method of claim 21, wherein the buffer is a Tris buffer.

24. The method of any one of claims 1-23, wherein the immunogenic composition does not comprise one or more of a saponin, a TLR4 agonist, and a liposome.

25. The method of any one of claims 1-23, wherein the immunogenic composition does not comprise one or more of QS21, 3-O-deacylated monophosphoryl lipid A (3D-MPL1), dioleoyl phosphatidylcholine, and cholesterol.

26. The method of any one of claims 1-25, wherein the gE antigen and the oligonucleotide are present in the immunogenic composition at a ratio of from 1:15 to 1:120 (wt/wt), optionally wherein the gE antigen and the oligonucleotide are present in the immunogenic composition at a ratio of from 1:15 to 1:60 (wt/wt).

27. The method of any one of claims 1-26, wherein the gE antigen and the oligonucleotide are present in the immunogenic composition at a ratio of about 1:15, about 1:20, about 1:30, about 1:40, about 1:60, about 1:80, or about 1:120 (wt/wt), optionally wherein the gE antigen and the oligonucleotide are present in the immunogenic composition at a ratio of about 1:30 (wt/wt) or about 1:60 (wt/wt).

28. The method of any one of claims 21-27, wherein the oligonucleotide and the Al3+ are present in the immunogenic composition at a ratio of from 8:1 to 2:1 (wt/wt), optionally wherein the oligonucleotide and the Al³⁺ are present in the immunogenic composition at a ratio of about 8:1 (wt/wt) or about 4:1 (wt/wt).

29. The method of any one of claims 1-28, wherein the human subject is 18 years of age or older.

30. The method of any one of claims 1-29, wherein the human subject is 50 years of age or older.

31. The method of claim 30, wherein the human subject is 50-69 years of age.

32. The method of any one of claims 1-31, wherein the human subject is suspected to have a latent VZV infection.

33. The method of any one of claims 1-32, wherein the human subject was not previously vaccinated against varicella or herpes zoster.

34. The method of any one of claims 6-33, wherein the human subject receives a first dose and a second dose of the immunogenic composition with the second dose administered from 1 month to 1 year after the first dose.

35. The method of claim 34, wherein the second dose is administered from 2 months to 6 months after the first dose.

36. The method of any one of claims 1-5 and 35, wherein the second dose is administered about 2 months after the first dose.

37. The method of any one of claims 1-36, wherein the immunogenic composition is administered by intramuscular injection.

38. The method of any one of claims 1-37, wherein the increase in cell-mediated immunity against VZV or the increase in immune response against VZV comprises an increase in frequency of gE antigen-reactive, activated CD4+ T cells, wherein the activated CD4+ T cells express two or more activation markers selected from interleukin-2, interferon-gamma, tumor necrosis factor-alpha, and CD40L (CD154).

39. The method of any one of claims 1-38, wherein administration of the immunogenic composition results in an increase in humoral immunity against VZV in the subject relative to humoral immunity against VZV in the subject prior to administration of the immunogenic composition.

40. The method of claim 39, wherein the increase in humoral immunity against VZV comprises an increased concentration of gE antigen-reactive IgG.

41. The method of claim 39 or claim 40, wherein the increase in humoral immunity against VZV comprises an increase in concentration of gE- and/or VZV-neutralizing antibodies.

42. The method of any one of claims 39-41, wherein the increase in cell-mediated immunity and the increase in humoral immunity are elicited in the subject by about two months after administration of the first dose of the immunogenic composition.

43. The method of any one of claims 39-41, wherein the increase in cell-mediated immunity and the increase in humoral immunity are elicited in the subject by about one month after administration of the second dose of the immunogenic composition.

44. The method of any one of claims 39-43, wherein the increase in cell-mediated immunity and the increase in humoral immunity are maintained in the subject for at least three months after administration of the second dose of the immunogenic composition.

45. The method of any one of claims 39-44, wherein administration of the immunogenic composition reduces risk of the subject developing herpes zoster relative to risk of a control subject who did not receive an immunogenic composition comprising the gE antigen.

46. The method of any one of claims 39-44, wherein administration of the immunogenic composition prevents the subject from developing herpes zoster, optionally for a period of at least 3 years after administration of the second dose of the immunogenic composition.

47. The method of any one of claims 39-44, wherein administration of the immunogenic composition prevents the subject from developing postherpetic neuralgia, optionally for a period of at least 3 years after administration of the second dose of the immunogenic composition.

48. The method of any one of claims 39-47, wherein the immunogenic composition is associated with a reduced risk of development of one or more solicited local reactions within 7 days following administration of the first dose and/or the second dose (day of vaccination and subsequent 6 days) relative to a comparator composition comprising the gE antigen and an AS01B adjuvant.

49. The method of claim 48, wherein the solicited local reactions comprise pain that prevents normal every day activities.

50. The method of any one of claims 39-49, wherein the immunogenic composition is associated with a reduced risk of development of one or more solicited systemic reactions within 7 days following administration of the first dose and/or the second dose (day of vaccination and subsequent 6 days) relative to a comparator composition comprising the gE antigen and an AS01B adjuvant.

51. The method of claim 50, wherein the solicited systemic reactions comprise fatigue, headache, and/or myalgia that prevents normal every day activities, and/or fever above 39° C.

52. The method of any one of claims 39-51, wherein the immunogenic composition is associated with a reduced risk of development of Guillain-Barre syndrome within 42 days following administration of the first dose and/or the second dose (day of vaccination and subsequent 41 days) relative to a comparator composition comprising the gE antigen and an AS01B adjuvant.

53. An immunogenic composition for use in a method of increasing cell-mediated immunity against varicella zoster virus (VZV) in a human subject in need thereof, the method comprising:
administering to the human subject the immunogenic composition, the immunogenic composition comprising: i) an unmethylated cytidine-phospho-guanosine (CpG)-containing oligonucleotide comprising the sequence of 5′-TGACTGTGAA CGTTCGAGAT GA-3′(SEQ ID NO: 1); and ii) a truncated VZV glycoprotein E (gE) antigen, which are present in the immunogenic composition in amounts effective to increase cell-mediated immunity against VZV relative to cell-mediated immunity against VZV in the subject prior to administration of the immunogenic composition, wherein a first dose and a second dose of the immunogenic composition is to be administered to the human subject, with the second dose to be administered from 2 months to 6 months after the first dose.

54. Use of an immunogenic composition in the manufacture of a medicament for increasing cell-mediated immunity against varicella zoster virus (VZV) in a human subject in need thereof, wherein:
the immunogenic composition comprises: i) an unmethylated cytidine-phospho-guanosine (CpG)-containing oligonucleotide comprising the sequence of 5′-TGACTGTGAA CGTTCGAGAT GA-3′(SEQ ID NO: 1); and ii) a truncated VZV glycoprotein E (gE) antigen, which are present in the immunogenic composition in amounts effective to increase cell-mediated immunity against VZV relative to cell-mediated immunity against VZV in the subject prior to administration of the immunogenic composition, and wherein a first dose and a second dose of the immunogenic composition is to be administered to the human subject, with the second dose to be administered from 2 months to 6 months after the first dose.

55. The composition or use of claim 53 or claim 54, wherein the immunogenic composition comprises: from about 25 μg to about 150 μg of the gE antigen, and from about 750 μg to about 6000 μg of the oligonucleotide.

56. The composition or use of claim 53 or claim 54, wherein the immunogenic composition comprises from about 50 μg to about 100 μg of the gE antigen, and from about 1500 μg to about 6000 μg of the oligonucleotide.

57. The composition or use of claim 53 or claim 54, wherein the immunogenic composition comprises about 25 μg, about 50 μg, about 75 μg, about 100 μg, about 125 μg, or about 150 μg of the gE antigen, and about 750 μg, about 1500 μg, about 3000 μg, or about 6000 μg of the oligonucleotide.

58. The composition or use of claim 57, wherein the immunogenic composition comprises about 100 μg of the gE antigen, and about 3000 μg or 6000 μg of the oligonucleotide.

59. An immunogenic composition for use in a method of increasing an immune response against varicella zoster virus (VZV) in a human subject in need thereof, the immunogenic composition comprising:
i) an unmethylated cytidine-phospho-guanosine (CpG)-containing oligonucleotide comprising the sequence of 5′-TGACTGTGAA CGTTCGAGAT GA-3′(SEQ ID NO: 1); and ii) a truncated VZV glycoprotein E (gE) antigen, wherein the immunogenic composition comprises about 100 μg of the gE antigen and about 3000 μg or about 6000 μg of the oligonucleotide.

60. Use of an immunogenic composition in the manufacture of a medicament for increasing an immune response against varicella zoster virus (VZV) in a human subject in need thereof, wherein:
the immunogenic composition comprises: i) an unmethylated cytidine-phospho-guanosine (CpG)-containing oligonucleotide comprising the sequence of 5′-TGACTGTGAA CGTTCGAGAT GA-3′(SEQ ID NO: 1); and ii) a truncated VZV glycoprotein E (gE) antigen, wherein the immunogenic composition comprises about 100 μg of the gE antigen and about 3000 μg or about 6000 μg of the oligonucleotide.

61. The composition or use of any one of claims 58-60, wherein the immunogenic composition comprises about 100 μg of the gE antigen, and about 3000 μg of the oligonucleotide.

62. The composition or use of any one of claims 58-60, wherein the immunogenic composition comprises about 100 μg of the gE antigen, and about 6000 μg of the oligonucleotide.

63. The composition or use of any one of claims 53-62, wherein the oligonucleotide comprises at least one phosphorothioate linkage, or wherein all nucleotide linkages are phosphorothioate linkages.

64. The composition or use of any one of claims 53-63, wherein the oligonucleotide is a single-stranded oligodeoxynucleotide.

65. The composition or use of any one of claims 53-64, wherein the gE antigen is a recombinant protein devoid of transmembrane and intravirion domains of a full-length VZV gE antigen.

66. The composition or use of claim 65, wherein the gE antigen comprises the amino acid sequence of SEQ ID NO:4, or an amino acid sequence that is at least 90%, 95%, 96%, 97%, 98% or 99% identical to SEQ ID NO:4.

67. The composition or use of claim 65, wherein the gE antigen comprises the amino acid sequence of SEQ ID NO:5, or an amino acid sequence that is at least 90%, 95%, 96%, 97%, 98% or 99% identical to SEQ ID NO:5.

68. The composition or use of any one of claims 65-67, wherein the recombinant protein is produced in mammalian cells.

69. The composition or use of any one of claims 53-68, wherein the immunogenic composition further comprises an aluminum salt adjuvant.

70. The composition or use of claim 69, wherein the aluminum salt adjuvant comprises one or more of the group consisting of amorphous aluminum hydroxyphosphate sulfate, aluminum hydroxide, aluminum phosphate, and potassium aluminum sulfate.

71. The composition or use of claim 69, wherein the aluminum salt adjuvant comprises aluminum hydroxide.

72. The composition or use of any one of claims 69-71, wherein the immunogenic composition comprises from about 0.25 mg to about 1.25 mg Al³⁺.

73. The composition or use of any one of claims 69-71, wherein the immunogenic composition comprises about 0.50 mg, about 0.75 mg, or about 1.00 mg Al³⁺.

74. The composition or use of claim 73, wherein the immunogenic composition comprises about 0.75 mg Al³⁺.

75. The composition or use of any one of claims 69-74, wherein the immunogenic composition further comprises a pharmaceutically acceptable buffer.

76. The composition or use of claim 75, wherein the buffer is not a phosphate-containing buffer.

77. The composition or use of claim 75, wherein the buffer is a Tris buffer.

78. The composition or use of any one of claims 53-77, wherein the immunogenic composition does not comprise one or more of a saponin, a TLR4 agonist, and a liposome.

79. The composition or use of any one of claims 53-77, wherein the immunogenic composition does not comprise one or more of QS21, 3-O-deacylated monophosphoryl lipid A (3D-MPL1), dioleoyl phosphatidylcholine, and cholesterol.

80. The composition or use of any one of claims 53-79, wherein the gE antigen and the oligonucleotide are present in the immunogenic composition at a ratio of from 1:15 to 1:120 (wt/wt), optionally wherein the gE antigen and the oligonucleotide are present in the immunogenic composition at a ratio of from 1:15 to 1:60 (wt/wt).

81. The composition or use of any one of claims 53-80, wherein the gE antigen and the oligonucleotide are present in the immunogenic composition at a ratio of about 1:15, about 1:20, about 1:30, about 1:40, about 1:60, about 1:80, or about 1:120 (wt/wt), optionally wherein the gE antigen and the oligonucleotide are present in the immunogenic composition at a ratio of about 1:30 (wt/wt) or about 1:60 (wt/wt).

82. The composition or use of any one of claims 75-81, wherein the oligonucleotide and the Al³⁺ are present in the immunogenic composition at a ratio of from 8:1 to 2:1 (wt/wt), optionally wherein the oligonucleotide and the Al³⁺ are present in the immunogenic composition at a ratio of about 8:1 (wt/wt) or about 4:1 (wt/wt).

83. The composition or use of any one of claims 53-82, wherein the human subject is 18 years of age or older.

84. The composition or use of any one of claims 53-83, wherein the human subject is 50 years of age or older.

85. The composition or use of claim 84, wherein the human subject is 50-69 years of age.

86. The composition or use of any one of claims 53-85, wherein the human subject is suspected to have a latent VZV infection.

87. The composition or use of any one of claims 53-86, wherein the human subject was not previously vaccinated against varicella or herpes zoster.

88. The composition or use of any one of claims 59-87, wherein the human subject is to receive a first dose and a second dose of the immunogenic composition with the second dose to be administered from 1 month to 1 year after the first dose.

89. The composition or use of claim 88, wherein the second dose is to be administered from 2 months to 6 months after the first dose.

90. The composition or use of any one of claims 53-58 and 89, wherein the second dose is to be administered about 2 months after the first dose.

91. The composition or use of any one of claims 53-90, wherein the immunogenic composition is to be administered by intramuscular injection.

92. The composition or use of any one of claims 53-91, wherein the increase in cell-mediated immunity against VZV or the increase in immune response against VZV comprises an increase in frequency of gE antigen-reactive, activated CD4+ T cells, wherein the activated CD4+ T cells express two or more activation markers selected from interleukin-2, interferon-gamma, tumor necrosis factor-alpha, and CD40L (CD154).

93. The composition or use of any one of claims 53-92, wherein administration of the immunogenic composition results in an increase in humoral immunity against VZV in the subject relative to humoral immunity against VZV in the subject prior to administration of the immunogenic composition.

94. The composition or use of claim 93, wherein the increase in humoral immunity against VZV comprises an increased concentration of gE antigen-reactive IgG.

95. The composition or use of claim 93 or claim 94, wherein the increase in humoral immunity against VZV comprises an increase in concentration of gE- and/or VZV-neutralizing antibodies.

96. The composition or use of any one of claims 93-95, wherein the increase in cell-mediated immunity and the increase in humoral immunity are elicited in the subject by about two months after administration of the first dose of the immunogenic composition.

97. The composition or use of any one of claims 93-95, wherein the increase in cell-mediated immunity and the increase in humoral immunity are elicited in the subject by about one month after administration of the second dose of the immunogenic composition.

98. The composition or use of any one of claims 93-97, wherein the increase in cell-mediated immunity and the increase in humoral immunity are maintained in the subject for at least three months after administration of the second dose of the immunogenic composition.

99. The composition or use of any one of claims 93-98, wherein administration of the immunogenic composition reduces risk of the subject developing herpes zoster relative to risk of a control subject who did not receive an immunogenic composition comprising the gE antigen.

100. The composition or use of any one of claims 93-98, wherein administration of the immunogenic composition prevents the subject from developing herpes zoster, optionally for a period of at least 3 years after administration of the second dose of the immunogenic composition.

101. The composition or use of any one of claims 93-98, wherein administration of the immunogenic composition prevents the subject from developing postherpetic neuralgia, optionally for a period of at least 3 years after administration of the second dose of the immunogenic composition.

102. The composition or use of any one of claims 93-101, wherein the immunogenic composition is associated with a reduced risk of development of one or more solicited local reactions within 7 days following administration of the first dose and/or the second dose (day of vaccination and subsequent 6 days) relative to a comparator composition comprising the gE antigen and an AS01B adjuvant.

103. The composition or use of claim 102, wherein the solicited local reactions comprise pain that prevents normal every day activities.

104. The composition or use of any one of claims 93-103, wherein the immunogenic composition is associated with a reduced risk of development of one or more solicited systemic reactions within 7 days following administration of the first dose and/or the second dose (day of vaccination and subsequent 6 days) relative to a comparator composition comprising the gE antigen and an AS01B adjuvant.

105. The composition or use of claim 104, wherein the solicited systemic reactions comprise fatigue, headache, and/or myalgia that prevents normal every day activities, and/or fever above 39° C.

106. The composition or use of any one of claims 93-105, wherein the immunogenic composition is associated with a reduced risk of development of Guillain-Barre syndrome within 42 days following administration of the first dose and/or the second dose (day of vaccination and subsequent 41 days) relative to a comparator composition comprising the gE antigen and an AS01B adjuvant.

107. An immunogenic composition comprising: i) an unmethylated cytidine-phospho-guanosine (CpG)-containing oligonucleotide comprising the sequence of 5′-TGACTGTGAA CGTTCGAGAT GA-3′(SEQ ID NO: 1); ii) a truncated VZV glycoprotein E (gE) antigen; and iii) at least one excipient,
wherein the gE antigen is a recombinant protein produced in mammalian cells and is present in the immunogenic composition in an amount of from about 25 μg to about 150 μg, the oligonucleotide is present in the immunogenic composition in an amount of from about 750 μg to about 6000 μg, and the at least one excipient comprises a pharmaceutically acceptable buffer.

108. An immunogenic composition comprising i) an unmethylated cytidine-phospho-guanosine (CpG)-containing oligonucleotide comprising the sequence of 5′-TGACTGTGAA CGTTCGAGAT GA-3′(SEQ ID NO: 1); ii) a truncated VZV glycoprotein E (gE) antigen; and iii) at least one excipient,
wherein the gE antigen is a recombinant protein produced in mammalian cells and is present in the immunogenic composition in an amount of about 100 μg, the oligonucleotide is present in the immunogenic composition in an amount of about 3000 μg or about 6000 μg, and the at least one excipient comprises a pharmaceutically acceptable buffer.

109. The immunogenic composition of claim 107 or claim 108, wherein the immunogenic composition comprises about 100 μg of the gE antigen, and about 3000 μg of the oligonucleotide.

110. The immunogenic composition of claim 107 or claim 108, wherein the immunogenic composition comprises about 100 μg of the gE antigen, and about 6000 μg of the oligonucleotide.

111. The immunogenic composition of any one of claims 107-110, wherein the gE antigen comprises the amino acid sequence of SEQ ID NO:4, or an amino acid sequence that is at least 90%, 95%, 96%, 97%, 98% or 99% identical to SEQ ID NO:4.

112. The immunogenic composition of any one of claims 107-110, wherein the gE antigen comprises the amino acid sequence of SEQ ID NO:5, or an amino acid sequence that is at least 90%, 95%, 96%, 97%, 98% or 99% identical to SEQ ID NO:5.

113. The immunogenic composition of any one of claims 107-112, further comprising an aluminum hydroxide adjuvant, wherein the immunogenic composition comprises from about 0.25 mg to about 1.25 mg Al³⁺.

114. The immunogenic composition of claim 113, wherein the immunogenic composition comprises about 0.75 mg Al³⁺.

115. The immunogenic composition of any one of claims 107-114, wherein the buffer is not a phosphate-containing buffer.

116. The immunogenic composition of any one of claims 107-115, wherein the buffer is a Tris buffer.
